(19) United States
(12) Patent Application Publication
(10) Pub. No.: US 2025/0099536 A1
(43) Pub. Date: Mar. 27, 2025

(54) PRISTINAMYCIN IA AND FLOPRISTIN COMBINATIONS IN TREATING OR PREVENTING BACTERIAL INFECTIONS

(71) Applicant: AIMMAX THERAPEUTICS, INC., DURHAM, NC (US)

(72) Inventors: KARA S. KEEDY, DURHAM, NC (US); LAURENE WANG, DURHAM, NC (US)

(73) Assignee: AIMMAX THERAPEUTICS, INC., DURHAM, NC (US)

(21) Appl. No.: 18/561,857

(22) PCT Filed: May 23, 2022
(86) PCT No.: PCT/US22/30552

§ 371 Date: Nov. 17, 2023

Related U.S. Application Data
(60) Provisional application No. 63/192,564, filed on May 24, 2021.

(51) Int. Cl. A61K 38/15; A61K 31/424; A61K 31/4353; A61P 31/04

(52) U.S. Cl. CPC A61K 38/15; A61K 31/424; A61K 31/4353; A61P 31/04

(57) ABSTRACT

The invention relates to the discovery of novel antibiotic compositions comprising various ratios of two streptogramins, streptogramin B (e.g., pristinamycin IA) and streptogramin A (e.g., flopristin), that possess synergistic antibacterial activities against Staphylococcus spp., Streptococcus spp., Enterococcus spp., Haemophilus spp., Moraxella spp., Legionella spp., Chlamydia spp., Mycoplasma spp., Ureaplasma spp., Neisseria spp., Gardnerella spp., Bacillus spp., and Francisella spp. as well as other bacterial species. In particular, the invention relates to pharmaceutical compositions of streptogramin B (e.g., pristinamycin IA) and streptogramin B (e.g., flopristin) in a ratio (pristinamycin IA flopristin) by weight ranging from about 0:100 to about 99:1. Furthermore, the invention relates to the pharmaceutical compositions and methods of treating and preventing a variety of bacterial infections.

### FIELD OF THE INVENTION

The invention relates to the discovery of novel streptogramin antibiotic compositions comprising various ratios of pristinamycin IA and flopristin that possess antibacterial activity. This includes activity against Staphylococcus spp., Streptococcus spp., Enterococcus spp., Haemophilus spp., Moraxella spp., Legionella spp., Chlamydia spp., Mycoplasma spp., Ureaplasma spp., Neisseria spp., Gardnerella spp., Bacillus spp., and Francisella spp. as well as other bacterial species. Additional aspects of the present invention include treating or preventing a variety of bacterial infections with various combinations of streptogramin B and streptogramin A compounds.

### BACKGROUND OF THE INVENTION

The introduction of antibiotics once reduced human morbidity and mortality caused by infectious diseases dramatically. However, the widespread use of antibiotics has led to the emergence of antibiotic resistant pathogenic bacteria leading to loss or reduction in clinical efficacy and consequently the need for new antibiotic therapeutic options. Mechanisms of antibiotic resistance include efflux of antibiotics by transporters, prevention of interaction of antibiotics with target by mutation, modification and protection of target, and modification of antibiotics (Ogawara H “Comparison of Antibiotic Resistance Mechanisms in Antibiotic-Producing and Pathogenic Bacteria” Molecules 2019, vol. 24, no. 19, 3430).

In 2019, the Centers for Disease Control and Prevention (CDC) released an update to their Antibiotic Resistance Threats in the United States report. The following pathogens were among those highlighted: drug-resistant Neisseria gonorrhoeae, methicillin-resistant Staphylococcus aureus (MRSA), drug-resistant Streptococcus pneumoniae, vancomycin-resistant Enterococci (VRE), erythromycin-resistant Streptococcus pyogenes, clindamycin-resistant Streptococcus agalactiae, and drug-resistant Mycoplasma genitalium (CDC: Antibiotic Resistance Threats in the United States, 2019. Atlanta, GA: CDC). N. gonorrhoeae, S. pneumoniae, MRSA, and VRE are also listed on the World Health Organizations (WHO) list of priority pathogens requiring new antibiotics (Tacconelli E, et al. “Discovery, research, and development of new antibiotics: the WHO priority list of antibiotic-resistant bacteria and tuberculosis” The Lancet Infectious Diseases 2018, vol. 18, no. 3, pages 318-327). Both of these lists serve to highlight public health priorities for the identification and development of new antibiotics.

Streptogramins are a unique class of antibiotics remarkable for their antibacterial activity and their mechanism of action. These antibiotics are produced naturally as secondary metabolites by several Streptomyces species. They consist of two structurally different compound groups. Streptogramins A or M are macrolactones (polyunsaturated macrocyclic lactones) of which pristinamycin IIA (PIIA) is an example; while streptogramins B or S are cyclic hexadepsipeptides of which pristinamycin IA (PIA) is an example.

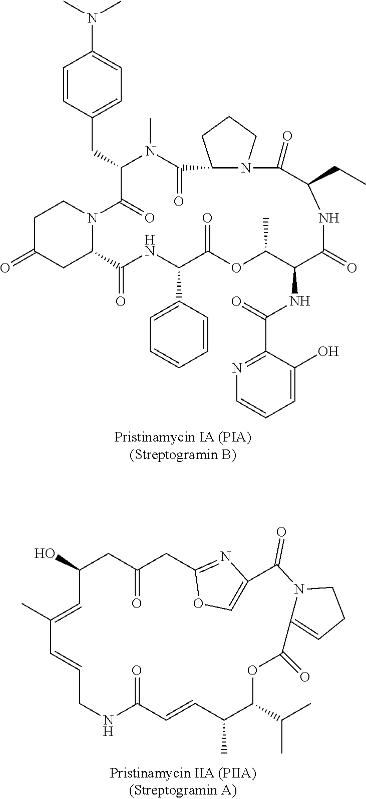

Streptogramins have demonstrated activity against gram-positive, certain gram-negative, and atypical bacteria as well as aerobic and anaerobic bacteria both in vitro and in vivo, including those with multi-drug resistance.

Both groups of streptogramins bind to bacterial ribosomes and inhibit protein synthesis and they act synergistically in vitro against many bacterial species.

Streptogramin A components inactivate the donor and acceptor sites of peptidyl transferases. They block two of the peptide chain elongation steps: aminoacyl-tRNA binding to the A site of ribosomes and peptide bond formation with peptidyl-tRNA at the P site.

Streptogramin B components have a more complex mechanism of action that involves inhibition of peptide bond formation with release of incomplete peptide chains.

The synergy between the streptogramin A and B components is believed to result from conformational changes imposed upon the peptidyl transferase center by the streptogramin A component and by inhibition of both early and late stages of protein synthesis. The conformational change increases ribosomal affinity for the streptogramins B component.

Two streptogramin antibiotic combinations have been approved for use in humans for the treatment of bacterial infections. Pristinamycin is a streptogramin antibiotic comprising pristinamycin IA and pristinamycin IIA co-produced by Streptomyces pristinaespiralis in a ratio by weight of about 30:70 and is used primarily in the treatment of staphylococcal infections, and to a lesser extent streptococcal infections. Pristinamycin is only available for use as a tablet for oral administration and due to its low oral bioavailability, it must be administered at high doses several times per day (1 g two to four times per day depending on infection type). Additionally, there is a need to improve efficacy of the composition as treatment failures have occurred.

Quinupristin-dalfopristin is the second streptogramin combination that has been approved for use in humans. The streptogramin B component, quinupristin, is a semi-synthetic derivative of pristinamycin IA and the streptogramin A component, dalfopristin, is a semi-synthetic derivative of pristinamycin IIA. The components are combined in a B to A ratio by weight of 30:70. It is available only in an intravenous injectable formulation.

A third but unapproved streptogramin combination, linopristin-flopristin, was evaluated in Phase 2 human clinical trials for bacterial skin and pneumonia infections. However, the development of this combination did not continue past clinical Phase 2. The streptogramin B component, linopristin, is a semisynthetic derivative of pristinamycin IA. The streptogramin A component, flopristin, is a semi-synthetic fluorinated derivative of pristinamycin IIB. This streptogramin combination has been studied clinically at a few different ratios, but the majority of studies have utilized a B to A ratio by weight of 30:70.

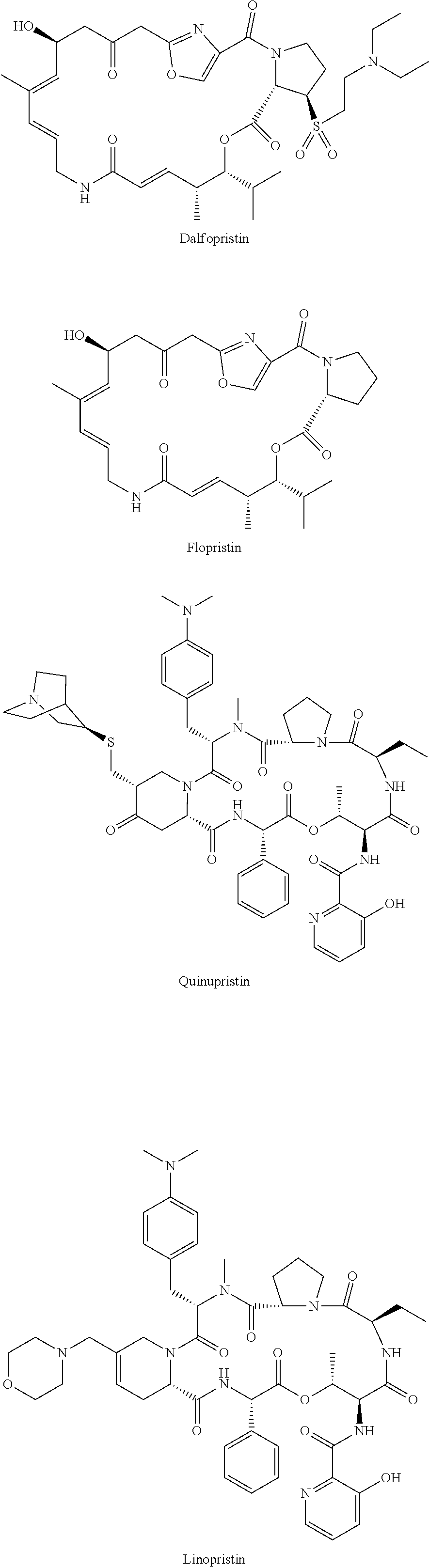

While streptogramins have been a useful class, improved streptogramin combinations with better potency and efficacy are desired to address the constant threat and evolution of antibiotic resistance. Further, more antibiotics with bactericidal activity are needed. Bactericidal activity is important to efficacy as a rapid elimination of the bacterial load during an infection reduces the potential for resistance development. By assessing the biological activities of different combinations of existing streptogramins, we have unexpectedly found a unique, novel combination of streptogramin B (i.e., pristinamycin IA) and streptogramin A (i.e., flopristin) consistently demonstrating the most potent activity among all other streptogramin combinations against a spectrum of important bacterial species.

### SUMMARY OF INVENTION

In one embodiment is provided a composition comprising a streptogramin B (e.g., pristinamycin IA or a pharmaceutically acceptable salt thereof) and a streptogramin A (e.g., flopristin or a pharmaceutically acceptable salt thereof), wherein the streptogramin B to streptogramin A (streptogramin B:streptogramin A) ratio by weight is about 1:99, about 2:98, about 3:97, about 4:96, about 5:95, about 6:94, about 7:93, about 8:92, about 9:91, about 10:90, about 11:89, about 12:88, about 13:87, about 14:86, about 15:85, about 16:84, about 17:83, about 18:82, about 19:81, about 20:80, about 21:79, about 22:78, about 23:77, about 24:76, about 25:75, about 26:74, about 27:73, about 28:72, about 29:71, about 30:70, about 31:69, about 32:68, about 33:67, about 34:66, about 35:65, about 36:64, about 37:63, about 38:62, about 39:61, about 40:60, about 41:59, about 42:58, about 43:57, about 44:56, about 45:55, about 46:54, about 47:53, about 48:52, about 49:51, about 50:50, about 51:49, about 52:48, about 53:47, about 54:46, about 55:45, about 56:44, about 57:43, about 58:42, about 59:41, about 60:40, about 61:39, about 62:38, about 63:37, about 64:36, about 65:35, about 66:34, about 67:33, about 68:32, about 69:31, about 70:30, about 71:29, about 72:28, about 73:27, about 74:26, about 75:25, about 76:24, about 77:23, about 78:22, about 79:21, about 80:20, about 81:19, about 82:18, about 83:17, about 84:16, about 85:15, about 86:14, about 87:13, about 88:12, about 89:11, about 90:10, about 91:9, about 92:8, about 93:7, about 94:6, about 95:5, about 96:4, about 97:3, about 98:2, or about 99:1.

In another embodiment is provided a composition comprising pristinamycin IA or a pharmaceutically acceptable salt thereof and flopristin or a pharmaceutically acceptable salt thereof, wherein the pristinamycin IA (PIA) to flopristin (PIA:flopristin) ratio by weight is about 1:99, about 2:98, about 3:97, about 4:96, about 5:95, about 6:94, about 7:93, about 8:92, about 9:91, about 10:90, about 11:89, about 12:88, about 13:87, about 14:86, about 15:85, about 16:84, about 17:83, about 18:82, about 19:81, about 20:80, about 21:79, about 22:78, about 23:77, about 24:76, about 25:75, about 26:74, about 27:73, about 28:72, about 29:71, about 30:70, about 31:69, about 32:68, about 33:67, about 34:66, about 35:65, about 36:64, about 37:63, about 38:62, about 39:61, about 40:60, about 41:59, about 42:58, about 43:57, about 44:56, about 45:55, about 46:54, about 47:53, about 48:52, about 49:51, about 50:50, about 51:49, about 52:48, about 53:47, about 54:46, about 55:45, about 56:44, about 57:43, about 58:42, about 59:41, about 60:40, about 61:39, about 62:38, about 63:37, about 64:36, about 65:35, about 66:34, about 67:33, about 68:32, about 69:31, about 70:30, about 71:29, about 72:28, about 73:27, about 74:26, about 75:25, about 76:24, about 77:23, about 78:22, about 79:21, about 80:20, about 81:19, about 82:18, about 83:17, about 84:16, about 85:15, about 86:14, about 87:13, about 88:12, about 89:11, about 90:10, about 91:9, about 92:8, about 93:7, about 94:6, about 95:5, about 96:4, about 97:3, about 98:2, or about 99:1.

In another embodiment is provided a pharmaceutical composition comprising pristinamycin IA or a pharmaceutically acceptable salt thereof and flopristin or a pharmaceutically acceptable salt thereof, wherein the pristinamycin IA (PIA) to flopristin (PIA:flopristin) ratio by weight is about 1:99, about 2:98, about 3:97, about 4:96, about 5:95, about 6:94, about 7:93, about 8:92, about 9:91, about 10:90, about 11:89, about 12:88, about 13:87, about 14:86, about 15:85, about 16:84, about 17:83, about 18:82, about 19:81, about 20:80, about 21:79, about 22:78, about 23:77, about 24:76, about 25:75, about 26:74, about 27:73, about 28:72, about 29:71, about 30:70, about 31:69, about 32:68, about 33:67, about 34:66, about 35:65, about 36:64, about 37:63, about 38:62, about 39:61, about 40:60, about 41:59, about 42:58, about 43:57, about 44:56, about 45:55, about 46:54, about 47:53, about 48:52, about 49:51, about 50:50, about 51:49, about 52:48, about 53:47, about 54:46, about 55:45, about 56:44, about 57:43, about 58:42, about 59:41, about 60:40, about 61:39, about 62:38, about 63:37, about 64:36, about 65:35, about 66:34, about 67:33, about 68:32, about 69:31, about 70:30, about 71:29, about 72:28, about 73:27, about 74:26, about 75:25, about 76:24, about 77:23, about 78:22, about 79:21, about 80:20, about 81:19, about 82:18, about 83:17, about 84:16, about 85:15, about 86:14, about 87:13, about 88:12, about 89:11, about 90:10, about 91:9, about 92:8, about 93:7, about 94:6, about 95:5, about 96:4, about 97:3, about 98:2, or about 99:1, and a pharmaceutically acceptable carrier or excipients.

In another embodiment is provided a kit comprising a first composition containing pristinamycin IA or a pharmaceutically acceptable salt thereof and a second composition containing flopristin or a pharmaceutically acceptable salt thereof, wherein the pristinamycin IA:flopristin ratio by weight is about 1:99, about 2:98, about 3:97, about 4:96, about 5:95, about 6:94, about 7:93, about 8:92, about 9:91, about 10:90, about 11:89, about 12:88, about 13:87, about 14:86, about 15:85, about 16:84, about 17:83, about 18:82, about 19:81, about 20:80, about 21:79, about 22:78, about 23:77, about 24:76, about 25:75, about 26:74, about 27:73, about 28:72, about 29:71, about 30:70, about 31:69, about 32:68, about 33:67, about 34:66, about 35:65, about 36:64, about 37:63, about 38:62, about 39:61, about 40:60, about 41:59, about 42:58, about 43:57, about 44:56, about 45:55, about 46:54, about 47:53, about 48:52, about 49:51, about 50:50, about 51:49, about 52:48, about 53:47, about 54:46, about 55:45, about 56:44, about 57:43, about 58:42, about 59:41, about 60:40, about 61:39, about 62:38, about 63:37, about 64:36, about 65:35, about 66:34, about 67:33, about 68:32, about 69:31, about 70:30, about 71:29, about 72:28, about 73:27, about 74:26, about 75:25, about 76:24, about 77:23, about 78:22, about 79:21, about 80:20, about 81:19, about 82:18, about 83:17, about 84:16, about 85:15, about 86:14, about 87:13, about 88:12, about 89:11, about 90:10, about 91:9, about 92:8, about 93:7, about 94:6, about 95:5, about 96:4, about 97:3, about 98:2, or about 99:1, wherein each composition further includes a pharmaceutically acceptable carrier or excipients, wherein the kit is optionally packaged as a blister pack, and wherein the kit includes instructions to administer to a subject in need thereof the first composition and the second composition at the same time, or to administer the first composition before or after the second composition is administered.

In another embodiment is provided a method of treating or preventing a bacterial infection in a subject in need thereof comprising administering to said subject a therapeutically effective amount of a pharmaceutical composition comprising pristinamycin IA or a pharmaceutically acceptable salt thereof and flopristin or a pharmaceutically acceptable salt thereof, wherein the pristinamycin IA:flopristin ratio by weight is about 1:99, about 2:98, about 3:97, about 4:96, about 5:95, about 6:94, about 7:93, about 8:92, about 9:91, about 10:90, about 11:89, about 12:88, about 13:87, about 14:86, about 15:85, about 16:84, about 17:83, about 18:82, about 19:81, about 20:80, about 21:79, about 22:78, about 23:77, about 24:76, about 25:75, about 26:74, about 27:73, about 28:72, about 29:71, about 30:70, about 31:69, about 32:68, about 33:67, about 34:66, about 35:65, about 36:64, about 37:63, about 38:62, about 39:61, about 40:60, about 41:59, about 42:58, about 43:57, about 44:56, about 45:55, about 46:54, about 47:53, about 48:52, about 49:51, about 50:50, about 51:49, about 52:48, about 53:47, about 54:46, about 55:45, about 56:44, about 57:43, about 58:42, about 59:41, about 60:40, about 61:39, about 62:38, about 63:37, about 64:36, about 65:35, about 66:34, about 67:33, about 68:32, about 69:31, about 70:30, about 71:29, about 72:28, about 73:27, about 74:26, about 75:25, about 76:24, about 77:23, about 78:22, about 79:21, about 80:20, about 81:19, about 82:18, about 83:17, about 84:16, about 85:15, about 86:14, about 87:13, about 88:12, about 89:11, about 90:10, about 91:9, about 92:8, about 93:7, about 94:6, about 95:5, about 96:4, about 97:3, about 98:2, or about 99:1, wherein the bacterial infection is an infection caused by one or more susceptible and/or resistant gram-positive bacteria, gram-negative bacteria, atypical bacteria, aerobic bacteria, and/or anaerobic bacteria, such as Bacillus spp., Bacteroides spp., Bordetella spp., Borrelia spp., Brucella spp., Burkholderia spp., Campylobacter spp., Chlamydia spp., Clostridium spp., Corynebacterium spp., Coxiella spp., Ehrlichia spp., Enterococcus spp., Francisella spp., Fusobacterium spp., Gardnerella spp., Haemophilus spp., Kingella spp., Legionella spp., Listeria spp., Moraxella spp., Mycoplasma spp., Mycobacterium spp., Neisseria spp., Nocardia spp., Peptostreptococcus spp., Porphyromonas spp., Propionibacterium spp., Prevotella spp., Rickettsia spp., Salmonella spp., Shigella spp., Staphylococcus spp., Streptococcus spp., Ureaplasma spp., Vibrio spp., or Yersinia spp.

Also described herein is a method of treating or preventing a respiratory infection and/or disease in a subject in need thereof, comprising administering to said subject a therapeutically effective amount of a pharmaceutical composition comprising pristinamycin IA or a pharmaceutically acceptable salt thereof and flopristin or a pharmaceutically acceptable salt thereof, wherein the pristinamycin IA:flopristin ratio by weight ranges from 1:99 to 99:1, wherein the respiratory infections and/or diseases comprise community acquired pneumonia or healthcare associated pneumonia, chronic bronchitis, sinusitis, acute maxillary sinusitis, acute exacerbations of chronic bronchitis, pharyngitis, chronic obstructive pulmonary disease, and non-tuberculosis mycobacteria infection caused by or associated with bacteria, such as one or more strains of susceptible and/or resistant Streptococcus pneumoniae, Streptococcus pyogenes, Staphylococcus aureus, Haemophilus influenzae, Haemophilus parainfluenzae, Moraxella catarrhalis, Legionella spp., Chlamydia pneumoniae, Mycoplasma pneumoniae, Bordetella pertussis, Neisseria gonorrhoeae, Corynebacterium diphtheriae, Coxiella burnettii, Peptostreptococcus spp., Fusobacterium spp., Bacteroides spp., Prevotella spp., Nocardia spp., or Mycobacterium spp.

Provided herein is a method of treating or preventing a sexually transmitted infection or a genitourinary tract infection in a subject in need thereof, comprising administering to said subject a therapeutically effective amount of a pharmaceutical composition comprising pristinamycin IA or a pharmaceutically acceptable salt thereof and flopristin or a pharmaceutically acceptable salt thereof, wherein the pristinamycin IA:flopristin ratio by weight ranges from 1:99 to 99:1, wherein the sexually transmitted infection or genitourinary tract infection is caused by or associated with bacteria, such as by one or more strains of susceptible and/or resistant Neisseria gonorrhoeae, Chlamydia trachomatis, Mycoplasma genitalium, Mycoplasma hominis, Ureaplasma urealyticum, Ureaplasma parvum, or Gardnerella vaginalis.

Described herein is a method of treating or preventing skin and/or soft tissue infections and/or disease in a subject in need thereof, comprising administering to said subject a therapeutically effective amount of a pharmaceutical composition comprising pristinamycin IA or a pharmaceutically acceptable salt thereof and flopristin or a pharmaceutically acceptable salt thereof, wherein the pristinamycin IA:flopristin ratio by weight ranges from 1:99 to 99:1, wherein the skin and/or soft tissue infections and/or disease is caused by bacteria, such as one or more strains of susceptible and/or resistant Staphylococcus aureus, Staphylococcus haemolyticus, Staphylococcus lugdunensis, Streptococcus agalactiae, viridans group streptococci, Streptococcus pyogenes, Enterococcus faecalis, Enterococcus faecium, Corynebacterium spp., or Propionibacterium acnes.

Described herein is a method of treating or preventing an infection in subjects with cystic fibrosis in need thereof comprising administering to said subject a therapeutically effective amount of pristinamycin IA or a pharmaceutically acceptable salt thereof and flopristin or a pharmaceutically acceptable salt thereof, wherein the pristinamycin IA:flopristin ratio by weight ranges from 1:99 to 99:1, wherein the infection or threat of infection is caused by or associated with bacteria, such as one or more strains of susceptible and/or resistant Staphylococcus aureus, Haemophilus influenzae, or Mycobacterium spp.

Described herein is a method of treating or preventing a bone and/or joint infection in a subject in need thereof comprising administering to said subject a therapeutically effective amount of a pharmaceutical composition comprising pristinamycin IA or a pharmaceutically acceptable salt thereof and flopristin or a pharmaceutically acceptable salt thereof, wherein the pristinamycin IA:flopristin ratio by weight ranges from 1:99 to 99:1, wherein the infection or threat of infection is caused by bacteria, such as one or more strains of susceptible and/or resistant Staphylococcus aureus, Staphylococcus haemolyticus, Staphylococcus lugdunensis, Streptococcus agalactiae, Streptococcus pyogenes, Streptococcus pneumoniae, viridans group streptococci, Salmonella spp., Shigella spp., Campylobacter spp., Yersinia spp., Enterococcus faecalis, Enterococcus faecium, Kingella kingae, Corynebacterium spp., Propionibacterium acnes, Chlamydia trachomatis or Neisseria gonorrhoeae.

Described herein is a method of treating or preventing endocarditis in a subject in need thereof, comprising administering to said subject a therapeutically effective amount of a pharmaceutical composition comprising pristinamycin IA or a pharmaceutically acceptable salt thereof and flopristin or a pharmaceutically acceptable salt thereof, wherein the pristinamycin IA:flopristin ratio by weight ranges from 1:99 to 99:1, wherein the infection or threat of infection is caused by bacteria, such as one or more strains of susceptible and/or resistant Staphylococcus aureus, Staphylococcus haemolyticus, Staphylococcus lugdunensis, Streptococcus gallolyticus, viridans group streptococci, Enterococcus faecalis, Enterococcus faecium, Haemophilus spp., Kingella spp., Legionella spp., or Corynebacterium spp.

Described herein is a method of treating or preventing bacteremia or sepsis in a subject in need thereof comprising administering to said subject a therapeutically effective amount of a pharmaceutical composition, comprising pristinamycin IA or a pharmaceutically acceptable salt thereof and flopristin or a pharmaceutically acceptable salt thereof, wherein the pristinamycin IA:flopristin ratio by weight ranges from 1:99 to 99:1, wherein the infection or threat of infection is caused by one or more strains of susceptible and/or resistant bacteria.

Disclosed herein is a method of treating or preventing anthrax, tularemia, plague, glanders, or meliodosis in a subject in need thereof, comprising administering to said subject a therapeutically effective amount of a pharmaceutical composition comprising pristinamycin IA or a pharmaceutically acceptable salt thereof and flopristin or a pharmaceutically acceptable salt thereof, wherein the pristinamycin IA:flopristin ratio by weight ranges from 1:99 to 99:1, wherein the infection or threat of infection is caused by bacteria, such as one or more strains of susceptible and/or resistant Bacillus anthracis, Francisella tularensis, Yersinia pestis, Burkholderia mallei, or Burkholderia pseudomallei.

Also described herein is a method of treating or preventing a bacterial infection comprising administering to a subject in need thereof a therapeutically effective amount of flopristin or a pharmaceutically acceptable salt thereof in a pharmaceutically acceptable carrier or excipients.

Disclosed herein is a method of treating or preventing a sexually transmitted infection or a genitourinary tract infection in a subject in need thereof comprising administering to said subject a therapeutically effective amount of a pharmaceutical composition comprising flopristin or a pharmaceutically acceptable salt thereof in a pharmaceutically acceptable carrier or excipients, wherein the sexually transmitted infection or genitourinary tract infection is caused by or associated with bacteria, such as by one or more strains of susceptible and/or resistant Neisseria gonorrhoeae, Chlamydia trachomatis, Mycoplasma genitalium, Mycoplasma hominis, Ureaplasma urealyticum, Ureaplasma parvum, or Gardnerella vaginalis.

In any of the methods for treating or preventing an infection or a condition described herein, the route of administration can be oral, rectal, transmucosal, inhalation, intranasal, topical (including dermal, ophthalmic, otic), vaginal, intestinal, buccal, or sublingual administration; parenteral delivery, including intramuscular, subcutaneous, intravenous, or intramedullary injections, as well as intrathecal, epidural, direct intraventricular, intraperitoneal, intraamniotic, intranasal, or intraocular injections or injection into the joints.

In any of the methods for treating or preventing an infection described herein, the subject can be an animal or human.

### BRIEF DESCRIPTION OF THE FIGURES

FIG. 1. shows the results of an example of checkerboard testing of the streptogramin B and streptogramin A components of the novel Combination of streptogramin B with streptogramin A #1 (CBA1; top), pristinamycin IA with pristinamycin IIA combination (middle), and linopristin with flopristin combination (bottom) against a strain of S. aureus. CBA1 is a combination of pristinamycin IA with flopristin. The shaded area represents wells with microbial growth. The MICs of specific streptogramin B:streptogramin A combination ratios are identified with bold borders surrounding the well.

FIG. 2. presents MIC distributions of CBA1 against various gram-positive, gram-negative, and atypical bacteria species compared to known streptogramin combinations pristinamycin IA-pristinamycin IIA, quinupristin-dalfopristin, and/or linopristin-flopristin. Lower values on X-axis indicate greater biological activity.

FIG. 3. is a graph depicting the time-to-kill kinetic activity of CBA1 against multidrug-resistant N. gonorrhoeae strain NCTC 13480 as compared to ceftriaxone. Bactericidal activity is defined a ≥3-log₁₀ colony forming unit (CFU)/mL reduction from baseline.

FIG. 4. shows the efficacy of CBA1 following intravenous (IV) or oral administration against S. aureus in the neutropenic thigh infection model. In class comparators included IV quinupristin-dalfopristin and oral pristinamycin IA-pristinamycin IIA. IV vancomycin and oral linezolid served as positive controls.

### DETAILED DESCRIPTION OF THE INVENTION

### Abbreviations and Definitions

The terms used in this specification generally have their ordinary meanings in the art, within the context of the invention, and in the specific context where each term is used. Certain terms that are used to describe the invention are discussed below, or elsewhere in the specification, to provide additional guidance to the practitioner regarding the description of the invention.

Unless otherwise defined, all technical and scientific terms used herein have the same meaning as commonly understood by one of ordinary skill in the art to which this invention pertains. In the case of conflict, the present document, including definitions will control.

The term “compound” or “agent” for streptogramins refers to a mixture of a streptogramin B component and a streptogramin A component in ratios by weight in the ranges specified.

The term “minimum inhibitory concentration” (MIC) refers to the lowest concentration of a chemical or compound which prevents visible growth of microorganisms including bacteria.

The term “minimum bactericidal concentration” (MBC) refers to the lowest concentration of an antibacterial agent that reduces the viability of the initial bacterial inoculum by ≥99.9%. It can be determined from broth dilution minimum inhibitory concentration (MIC) tests by subculturing to agar plates that do not contain the test agent.

The terms “treating” or “treatment” refer to administration of an effective amount of a therapeutic agent to a subject in need thereof with the purpose of cure, alleviate, relieve, remedy, and/or ameliorate the disease. Such a subject can be identified by a health care professional based on results from any suitable diagnostic method. “Treating” or “treatment” also includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, delay or slowing of disease progression, amelioration or palliation of the disease state, diminishment of the reoccurrence of disease, and remission, whether partial or total and whether detectable or undetectable.

The terms “prevent,” “preventative,” “preventive,” or “prophylactic” refers to a treatment that decreases the occurrence of disease symptoms (e.g., infectious disease symptoms) in a subject. Prevent, preventative, preventive or prophylactic may be complete (no detectable symptoms) or partial, such that fewer symptoms are observed than would likely occur absence of a preventive treatment. Preventive treatment also refers to pretreating a subject prior to exposure to an infectious agent.

The terms “synergy” or “synergism” or “synergistic” refer to an interaction between two or more agents or drugs that causes the total effect of the combined agents or drugs to be greater than the sum of the individual effects of each agent or drug.

The term “additive effect” refers to when the combined effect of two or more agents or drugs is equal to the sum of the effect of each agent or drug given alone.

The term “susceptible and/or resistant” bacteria strain refers to bacteria that are susceptible, meaning there is a high likelihood of therapeutic success using a dosing regimen of the agent, and/or resistant, meaning there is a high likelihood of therapeutic failure using a dosing regimen of the agent.

The term “known” streptogramins combinations refers to the three streptogramin combinations that have been approved for human use or tested in human clinical studies, which include combinations of pristinamycin IA with pristinamycin IIA, quinupristin with dalfopristin, and linopristin with flopristin.

The terms “administer,” “administering”, “administration,” and the like, as used herein, refer to the methods that may be used to enable delivery of agents or compositions to the desired site of biological action.

The term “subject” refers to an animal, preferably a mammal, and most preferably a human, who is the object of treatment, observation or experiment. Exemplary mammals include mice, rats, rodents, hamsters, gerbils, rabbits, guinea pigs, dogs, cats, sheep, goats, cows, horses, giraffes, elephants, tigers, lions, bears, platypuses, primates, such as monkeys, chimpanzees, apes, and humans. An animal can also be a bird exemplified as chicken, turkey and other bird species.

The phrase “pharmaceutically acceptable” is employed herein to refer to those compounds, materials, compositions, formulations and/or dosage forms which are, within the scope of sound medical judgment, suitable for use in contact with the tissues of human beings or, as the case may be, an animal without excessive toxicity, irritation, allergic response. or other problem or complication, commensurate with a reasonable benefit/risk ratio.

The term “pharmaceutical composition” refers to a mixture of a compound or compounds disclosed herein with other chemical components such as diluents, binders, excipients or carriers to make a dosage form. The pharmaceutical composition facilitates administration of the compound(s) to a subject.

The term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

As used herein, the amount, dose, dosage or concentration of any pharmaceutical composition comprising pristinamycin IA or a pharmaceutical acceptable salt thereof and flopristin or a pharmaceutical acceptable salt thereof is the sum of the amounts or concentrations of the free base of pristinamycin IA and flopristin; and the amount, dose, dosage or concentration of any pharmaceutical composition comprising pristinamycin IA or a pharmaceutical acceptable salt thereof or flopristin or a pharmaceutical acceptable salt thereof is the amount of the free base of pristinamycin IA or flopristin, respectively.

### Composition

As a general proposition, a therapeutically effective amount or prophylactically effective amount of a streptogramin B (e.g., pristinamycin IA or a pharmaceutically acceptable salt thereof) and a streptogramin A (e.g., flopristin or a pharmaceutically acceptable salt thereof) either together within a single pharmaceutical composition (dosage form) or separately in two independent pharmaceutical compositions (dosage forms), e.g., in a kit, are provided for treating or preventing a bacterial infection or a condition caused by a bacterial infection.

As a general proposition, in an embodiment, a therapeutically effective amount or prophylactically effective amount of any pharmaceutical composition disclosed herein can be administered in a range from about 10 ng/kg body weight/day to about 100 mg/kg body weight/day whether by one or more administrations. In a particular embodiment, a therapeutic compound is administered in the range of from about 10 ng/kg body weight/day to about 100 mg/kg body weight/day, about 10 ng/kg body weight/day to about 10 mg/kg body weight/day, about 10 ng/kg body weight/day to about 1 mg/kg body weight/day, about 10 ng/kg body weight/day to about 100 μg/kg body weight/day, about 10 ng/kg body weight/day to about 10 μg/kg body weight/day, about 10 ng/kg body weight/day to about 1 μg/kg body weight/day, about 10 ng/kg body weight/day to about 100 ng/kg body weight/day, about 100 ng/kg body weight/day to about 100 mg/kg body weight/day, about 100 ng/kg body weight/day to about 10 mg/kg body weight/day, about 100 ng/kg body weight/day to about 1 mg/kg body weight/day, about 100 ng/kg body weight/day to about 100 μg/kg body weight/day, about 100 ng/kg body weight/day to about 10 μg/kg body weight/day, about 100 ng/kg body weight/day to about 1 μg/kg body weight/day, about 1 μg/kg body weight/day to about 100 mg/kg body weight/day, 1 μg/kg body weight/day to about 10 mg/kg body weight/day, about 1 μg/kg body weight/day to about 1 mg/kg body weight/day, about 1 μg/kg body weight/day to about 100 μg/kg body weight/day, about 1 μg/kg body weight/day to about 10 μg/kg body weight/day, about 10 μg/kg body weight/day to about 100 mg/kg body weight/day, about 10 μg/kg body weight/day to about 10 mg/kg body weight/day, about 10 μg/kg body weight/day to about 1 mg/kg body weight/day, about 10 μg/kg body weight/day to about 100 μg/kg body weight/day, about 100 μg/kg body weight/day to about 100 mg/kg body weight/day, about 100 μg/kg body weight/day to about 10 mg/kg body weight/day, about 100 μg/kg body weight/day to about 1 mg/kg body weight/day, about 1 mg/kg body weight/day to about 100 mg/kg body weight/day, about 1 mg/kg body weight/day to about 10 mg/kg body weight/day, about 10 mg/kg body weight/day to about 100 mg/kg body weight/day.

In some embodiments, a therapeutically effective amount or prophylactically effective amount of any pharmaceutical composition disclosed herein is administered in the range from about 100 ng to about 1 μg per individual administration, from about 100 ng to about 10 μg per individual administration, from about 100 ng to about 100 μg per individual administration, from about 100 ng to about 1 mg per individual administration, about 100 ng to about 10 mg per individual administration, about 100 ng to about 100 mg per individual administration, about 100 ng to about 1000 mg per individual administration, about 100 ng to about 10,000 mg per individual administration, from about 1 μg to about 10 μg per individual administration, about 1 μg to about 100 μg per individual administration, about 1 μg to about 1 mg per individual administration, about 1 μg to about 10 mg per individual administration, about 1 μg to about 100 mg per individual administration, about 1 μg to about 1000 mg per individual administration, about 1 μg to about 10,000 mg per individual administration, from about 10 μg to about 100 μg per individual administration, about 10 μg to about 1 mg per individual administration, about 10 μg to about 10 mg per individual administration, about 10 μg to about 100 mg per individual administration, about 10 μg to about 1000 mg per individual administration, about 10 μg to about 10,000 mg per individual administration, from about 100 μg to about 1 mg per individual administration, about 100 μg to about 10 mg per individual administration, about 100 μg to about 100 mg per individual administration, about 100 μg to about 1000 mg per individual administration, about 100 μg to about 10,000 mg per individual administration, from about 1 mg to about 10 mg per individual administration, about 1 mg to about 100 mg per individual administration, about 1 mg to about 1000 mg per individual administration, about 1 mg to about 10,000 mg per individual administration, from about 10 mg to about 100 mg per individual administration, about 10 mg to about 1000 mg per individual administration, about 10 mg to about 10,000 mg per individual administration, from about 100 mg to about 1000 mg per individual administration, about 100 mg to about 10,000 mg per individual administration, or from about 1000 mg to about 10,000 mg per individual administration. The composition may be administered once, twice, three times, four times, six times, or eight times daily, and every 1, 2, 3, 4, 5, 6 or 7 days, or every 1, 2, 3 or 4 weeks.

In some embodiments, any pharmaceutical composition disclosed herein reduces an infection or prevents the occurrence of an infection by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%.

In some embodiments, any pharmaceutical composition disclosed herein reduces or prevents the occurrence of an infection by, e.g., about 10%, about 15%, about 20%, about 25%, about 30%, about 35%, about 40%, about 45%, about 50%, about 55%, about 60%, about 65%, about 70%, about 75%, about 80%, about 85%, about 90%, about 95% or about 100%.

In yet other aspects of this embodiment, any pharmaceutical composition disclosed herein reduces an infection by a range of, e.g., about 5% to about 100%, about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, about 60% to about 80%, about 10% to about 70%, about 20% to about 70%, about 30% to about 70%, about 40% to about 70%, or about 50% to about 70%.

A pharmaceutical composition comprising pristinamycin IA or a pharmaceutically acceptable salt thereof and/or flopristin or a pharmaceutically acceptable salt thereof disclosed herein is in an amount sufficient to allow flexibility and customary administration to a subject.

In some embodiments, a therapeutically effective amount or prophylactically effective amount of any pharmaceutical composition disclosed herein may be, e.g., at least 5 mg, at least 10 mg, at least 15 mg, at least 20 mg, at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, at least 100 mg, at least 125 mg, at least 150 mg, at least 175 mg, at least 200 mg, at least 250 mg, at least 300 mg, at least 350 mg, at least 400 mg, at least 450 mg, at least 500 mg, at least 550 mg, at least 600 mg, at least 650 mg, at least 700 mg, at least 750 mg, at least 800 mg, at least 850 mg, at least 900 mg, at least 950 mg, at least 1,000 mg, at least 1,100 mg, at least 1,200 mg, at least 1,300 mg, at least 1,400 mg, at least 1,500 mg, at least 1,600 mg, at least 1,700 mg, at least 1,800 mg, at least 1,900 mg, or at least 2,000 mg of a pharmaceutical composition.

In some embodiments, a therapeutically effective amount or prophylactically effective amount of any pharmaceutical composition disclosed herein may be in the range of, e.g., about 5 mg to about 100 mg, about 10 mg to about 100 mg, about 50 mg to about 150 mg, about 100 mg to about 250 mg, about 150 mg to about 350 mg, about 250 mg to about 500 mg, about 350 mg to about 600 mg, about 500 mg to about 750 mg, about 600 mg to about 900 mg, about 750 mg to about 1,000 mg, about 850 mg to about 1,200 mg, about 1,000 mg to about 1,500 mg, or about 1,200 mg to about 2,000 mg. In still other aspects of this embodiment, a pharmaceutical composition disclosed herein may be in the range of, e.g., about 5 mg to about 250 mg, about 5 mg to about 500 mg, about 5 mg to about 750 mg, about 5 mg to about 1,000 mg, about 5 mg to about 1,500 mg, about 5 mg to about 2,000 mg, about 10 mg to about 250 mg, about 10 mg to about 500 mg, about 10 mg to about 750 mg, about 10 mg to about 1,000 mg, about 10 mg to about 1,500 mg, about 10 mg to about 2,000 mg, about 50 mg to about 250 mg, about 50 mg to about 500 mg, about 50 mg to about 750 mg, about 50 mg to about 1,000 mg, about 50 mg to about 1,500 mg, about 50 mg to about 2,000 mg, about 100 mg to about 250 mg, about 100 mg to about 500 mg, about 100 mg to about 750 mg, about 100 mg to about 1,000 mg, about 100 mg to about 1,500 mg, about 100 mg to about 2,000 mg, about 250 mg to about 500 mg, about 250 mg to about 750 mg, about 250 mg to about 1,000 mg, about 250 mg to about 1,500 mg, about 250 mg to about 2,000 mg, about 500 mg to about 750 mg, about 500 mg to about 1,000 mg, about 500 mg to about 1,500 mg, about 500 mg to about 2,000 mg, about 750 mg to about 1,000 mg, about 750 mg to about 1,500 mg, or about 750 mg to about 2,000 mg.

A therapeutically effective amount or prophylactically effective amount of any pharmaceutical composition disclosed herein may comprise a solvent, emulsion, vehicle, carrier or a diluent in an amount of, e.g., less than about 90% (v/v), less than about 85% (v/v), less than about 80% (v/v), less than about 75% (v/v), less than about 70% (v/v), less than about 65% (v/v), less than about 60% (v/v), less than about 55% (v/v), less than about 50% (v/v), less than about 45% (v/v), less than about 40% (v/v), less than about 35% (v/v), less than about 30% (v/v), less than about 25% (v/v), less than about 20% (v/v), less than about 15% (v/v), less than about 10% (v/v), less than about 5% (v/v), or less than about 1% (v/v).

A therapeutically effective amount or prophylactically effective amount of any pharmaceutical composition disclosed herein may comprise a solvent, emulsion, vehicle, carrier or other diluent in an amount in a range of, e.g., about 1% (v/v) to 90% (v/v), about 1% (v/v) to 80% (v/v), about 1% (v/v) to 70% (v/v), about 1% (v/v) to 60% (v/v), about 1% (v/v) to 50% (v/v), about 1% (v/v) to 40% (v/v), about 1% (v/v) to 30% (v/v), about 1% (v/v) to 20% (v/v), about 1% (v/v) to 10% (v/v), about 2% (v/v) to 50% (v/v), about 2% (v/v) to 40% (v/v), about 2% (v/v) to 30% (v/v), about 2% (v/v) to 20% (v/v), about 2% (v/v) to 10% (v/v), about 4% (v/v) to 50% (v/v), about 4% (v/v) to 40% (v/v), about 4% (v/v) to 30% (v/v), about 4% (v/v) to 20% (v/v), about 4% (v/v) to 10% (v/v), about 6% (v/v) to 50% (v/v), about 6% (v/v) to 40% (v/v), about 6% (v/v) to 30% (v/v), about 6% (v/v) to 20% (v/v), about 6% (v/v) to 10% (v/v), about 8% (v/v) to 50% (v/v), about 8% (v/v) to 40% (v/v), about 8% (v/v) to 30% (v/v), about 8% (v/v) to 20% (v/v), about 8% (v/v) to 15% (v/v), or about 8% (v/v) to 12% (v/v), about 25% (v/v) to 75% (v/v), about 30% (v/v) to 60% (v/v), about 20% (v/v) to 80% (v/v), about 40% (v/v) to 60% (v/v), about 45% (v/v) to 55% (v/v), about 35% (v/v) to 65% (v/v), about 50% (v/v) to 90% (v/v), about 45% (v/v) to 60% (v/v), about 15% (v/v) to 75% (v/v), about 20% (v/v) to 65% (v/v), or about 25% (v/v) to 50%.

The final concentration of any therapeutically effective or prophylactically effective pharmaceutical composition disclosed herein may be of any concentration desired. In an aspect of this embodiment, the final concentration of a pharmaceutical composition comprising pristinamycin IA or a pharmaceutically acceptable salt thereof and/or flopristin or a pharmaceutically acceptable salt thereof may be therapeutically effective. The final concentration of the active ingredient in any pharmaceutical composition disclosed herein may be, e.g., at least 0.01 mg/mL, at least 0.1 mg/mL, at least 1 mg/mL, at least 10 mg/mL, at least 25 mg/mL, at least 50 mg/mL, at least 100 mg/mL, at least 200 mg/mL at least 500 mg/mL or at least 1000 mg/mL. In other aspects of this embodiment, the final concentration of a pharmaceutical composition in a pharmaceutical composition may be in a range of, e.g., about 0.01 mg/mL to about 1,000 mg/mL, about 0.1 mg/mL to about 1,000 mg/mL, about 1 mg/mL to about 1,000 mg/mL, about 10 mg/mL to about 1000 mg/mL, about 25 mg/mL to about 1000 mg/mL, about 50 mg/mL to about 1000 mg/mL, about 100 mg/mL to about 1,000 mg/mL, about 250 mg/mL to about 1000 mg/mL, about 500 mg/mL to about 1,000 mg/mL, about 750 mg/mL to about 1,000 mg/mL, about 0.01 mg/mL to about 750 mg/mL, about 0.1 mg/mL to about 750 mg/mL, about 1 mg/mL to about 750 mg/mL, about 10 mg/mL to about 750 mg/mL, about 25 mg/mL to about 750 mg/mL, about 50 mg/mL to about 750 mg/mL, about 100 mg/mL to about 750 mg/mL, about 250 mg/mL to about 750 mg/mL, about 500 mg/mL to about 750 mg/mL, about 0.01 mg/mL to about 500 mg/mL, about 0.1 mg/mL to about 500 mg/mL, about 1 mg/mL to about 500 mg/mL, about 10 mg/mL to about 500 mg/mL, about 25 mg/mL to about 500 mg/mL, about 50 mg/mL to about 500 mg/mL, about 100 mg/mL to about 500 mg/mL, about 250 mg/mL to about 500 mg/mL, about 0.01 mg/mL to about 250 mg/mL, about 0.1 mg/mL to about 250 mg/mL, about 1 mg/mL to about 250 mg/mL, about 10 mg/mL to about 250 mg/mL, about 25 mg/mL to about 250 mg/mL, about 50 mg/mL to about 250 mg/mL, about 100 mg/mL to about 250 mg/mL, about 0.01 mg/mL to about 100 mg/mL, about 0.1 mg/mL to about 100 mg/mL, about 1 mg/mL to about 100 mg/mL, about 10 mg/mL to about 100 mg/mL, about 25 mg/mL to about 100 mg/mL, about 50 mg/mL to about 100 mg/mL, about 0.01 mg/mL to about 50 mg/mL, about 0.1 mg/mL to about 50 mg/mL, about 1 mg/mL to about 50 mg/mL, about 10 mg/mL to about 50 mg/mL, about 25 mg/mL to about 50 mg/mL, about 0.01 mg/mL to about 25 mg/mL, about 0.1 mg/mL to about 25 mg/mL, about 1 mg/mL to about 25 mg/mL, about 10 mg/mL to about 25 mg/mL, about 0.01 mg/mL to about 10 mg/mL, about 0.1 mg/mL to about 10 mg/mL, about 1 mg/mL to about 10 mg/mL, about 0.01 mg/mL to about 1 mg/mL, about 0.1 mg/mL to about 1 mg/mL, or about 0.01 mg/mL to about 0.1 mg/mL.

Aspects of the present specification disclose, in part, treating a subject suffering from a bacterial infection. As used herein, the term “treating,” refers to reducing or eliminating in a subject a clinical symptom of bacterial infection; or delaying or preventing in a subject the onset of a clinical symptom of a bacterial infection. For example, the term “treating” can mean reducing a sign or symptom of a condition characterized by a bacterial infection by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% at least 95%, or at least 100%. The actual symptoms associated with bacterial infection are well known and can be determined by a person of ordinary skill in the art by taking into account factors, including, without limitation, the location of the bacterial infection, the cause of the bacterial infection, the severity of the bacterial infection, and/or the tissue or organ affected by the bacterial infection. Those of skill in the art will know the appropriate symptoms or indicators associated with a specific type of bacterial infection and will know how to determine if a subject is a candidate for treatment as disclosed herein.

A therapeutically effective amount of any pharmaceutical composition disclosed herein can reduce a symptom associated with bacterial infection by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%. In other aspects of this embodiment, a therapeutically effective amount of a pharmaceutical composition disclosed herein reduces a symptom associated with bacterial infection by, e.g., at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% or at most 100%. In yet other aspects of this embodiment, a therapeutically effective amount of a pharmaceutical composition comprising pristinamycin IA or a pharmaceutically acceptable salt thereof and flopristin or a pharmaceutically acceptable salt thereof disclosed herein reduces a symptom associated with bacterial infection by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

A therapeutically effective amount of any pharmaceutical composition disclosed herein generally may be in the range of about 10 ng/kg/day to about 100 mg/kg/day. The pharmaceutical composition is administered, for example, every 1, 2, 3, 4, 5, 6 or 7 days, or every 1, 2, 3 or 4 weeks. An effective amount of any pharmaceutical composition disclosed herein may be, e.g., at least 10 ng/kg/day, at least at least 0.1 mg/kg/day, at least 1.0 mg/kg/day, at least 5.0 mg/kg/day, at least 10 mg/kg/day, at least 15 mg/kg/day, at least 20 mg/kg/day, at least 25 mg/kg/day, at least 30 mg/kg/day, at least 35 mg/kg/day, at least 40 mg/kg/day, at least 45 mg/kg/day, or at least 50 mg/kg/day and administered, for example, every 1, 2, 3, 4, 5, 6 or 7 days, or every 1, 2, 3 or 4 weeks. An effective amount of any pharmaceutical composition disclosed herein may be in the range of, e.g., about 10 ng/kg/day to about 10 mg/kg/day, about 10 ng/kg/day to about 15 mg/kg/day, about 10 ng/kg/day to about 20 mg/kg/day, about 10 ng/kg/day to about 25 mg/kg/day, about 10 ng/kg/day to about 30 mg/kg/day, about 10 ng/kg/day to about 35 mg/kg/day, about 10 ng/kg/day to about 40 mg/kg/day, about 10 ng/kg/day to about 45 mg/kg/day, about 10 ng/kg/day to about 50 mg/kg/day, about 10 ng/kg/day to about 75 mg/kg/day, or about 10 ng/kg/day to about 100 mg/kg/day and administered, for example, every 1, 2, 3, 4, 5, 6 or 7 days, or every 1, 2, 3 or 4 weeks. An effective amount of any pharmaceutical composition disclosed herein may be in the range of, e.g., about 0.1 mg/kg/day to about 10 mg/kg/day, about 0.1 mg/kg/day to about 15 mg/kg/day, about 0.1 mg/kg/day to about 20 mg/kg/day, about 0.1 mg/kg/day to about 25 mg/kg/day, about 0.1 mg/kg/day to about 30 mg/kg/day, about 0.1 mg/kg/day to about 35 mg/kg/day, about 0.1 mg/kg/day to about 40 mg/kg/day, about 0.1 mg/kg/day to about 45 mg/kg/day, about 0.1 mg/kg/day to about 50 mg/kg/day, about 0.1 mg/kg/day to about 75 mg/kg/day, or about 0.1 mg/kg/day to about 100 mg/kg/day and administered, for example, every 1, 2, 3, 4, 5, 6 or 7 days, or every 1, 2, 3 or 4 weeks. An effective amount of any pharmaceutical composition disclosed herein may be in the range of, e.g., about 1 mg/kg/day to about 10 mg/kg/day, about 1 mg/kg/day to about 15 mg/kg/day, about 1 mg/kg/day to about 20 mg/kg/day, about 1 mg/kg/day to about 25 mg/kg/day, about 1 mg/kg/day to about 30 mg/kg/day, about 1 mg/kg/day to about 35 mg/kg/day, about 1 mg/kg/day to about 40 mg/kg/day, about 1 mg/kg/day to about 45 mg/kg/day, about 1 mg/kg/day to about 50 mg/kg/day, about 1 mg/kg/day to about 75 mg/kg/day, or about 1 mg/kg/day to about 100 mg/kg/day and administered, for example, every 1, 2, 3, 4, 5, 6 or 7 days, or every 1, 2, 3 or 4 weeks.

In liquid, suspension, and semi-solid formulations, a concentration of any therapeutic composition disclosed herein typically may be between about 0.01 mg/mL to about 1,000 mg/mL. A therapeutically effective amount of any pharmaceutical composition disclosed herein may be from, e.g., about 0.01 mg/mL to about 1,000 mg/mL, about 0.1 mg/mL to about 1,000 mg/mL, about 1 mg/mL to about 1,000 mg/mL, about 10 mg/mL to about 1000 mg/mL, about 25 mg/mL to about 1000 mg/mL, about 50 mg/mL to about 1000 mg/mL, about 100 mg/mL to about 1,000 mg/mL, about 200 mg/mL to about 1000 mg/mL, about 500 mg/mL to about 1,000 mg/mL, about 750 mg/mL to about 1,000 mg/mL, about 1 mg/mL to about 10 mg/mL, about 1 mg/mL to about 50 mg/mL, about 1 mg/mL to about 100 mg/mL, about 1 mg/mL to about 200 mg/mL, about 1 mg/mL to about 300 mg/mL, about 1 mg/mL to about 400 mg/mL, about 1 mg/mL to about 500 mg/mL, about 1 mg/mL to about 600 mg/mL, about 1 mg/mL to about 700 mg/mL, about 1 mg/mL to about 800 mg/mL, about 1 mg/mL to about 900 mg/mL, about 1 mg/mL to about 1,000 mg/mL, about 10 mg/mL to about 50 mg/mL, about 10 mg/mL to about 100 mg/mL, about 10 mg/mL to about 200 mg/mL, about 10 mg/mL to about 300 mg/mL, about 10 mg/mL to about 400 mg/mL, about 10 mg/mL to about 500 mg/mL, about 10 mg/mL to about 600 mg/mL, about 10 mg/mL to about 700 mg/mL, about 10 mg/mL to about 800 mg/mL, about 10 mg/mL to about 900 mg/mL, about 10 mg/mL to about 1,000 mg/mL, about 50 mg/mL to about 100 mg/mL, about 50 mg/mL to about 200 mg/mL, about 50 mg/mL to about 300 mg/mL, about 50 mg/mL to about 400 mg/mL, about 50 mg/mL to about 500 mg/mL, about 50 mg/mL to about 600 mg/mL, about 50 mg/mL to about 700 mg/mL, about 50 mg/mL to about 800 mg/mL, about 50 mg/mL to about 900 mg/mL, about 50 mg/mL to about 1,000 mg/mL, about 100 mg/mL to about 200 mg/mL, about 100 mg/mL to about 300 mg/mL, about 100 mg/mL to about 400 mg/mL, about 100 mg/mL to about 500 mg/mL, about 100 mg/mL to about 600 mg/mL, about 100 mg/mL to about 700 mg/mL, about 100 mg/mL to about 800 mg/mL, about 100 mg/mL to about 900 mg/mL, about 100 mg/mL to about 1,000 mg/mL, about 200 mg/mL to about 300 mg/mL, about 200 mg/mL to about 400 mg/mL, about 200 mg/mL to about 500 mg/mL, about 200 mg/mL to about 600 mg/mL, about 200 mg/mL to about 700 mg/mL, about 200 mg/mL to about 800 mg/mL, about 200 mg/mL to about 900 mg/mL, about 200 mg/mL to about 1,000 mg/mL, about 300 mg/mL to about 400 mg/mL, about 300 mg/mL to about 500 mg/mL, about 300 mg/mL to about 600 mg/mL, about 300 mg/mL to about 700 mg/mL, about 300 mg/mL to about 800 mg/mL, about 300 mg/mL to about 900 mg/mL, about 300 mg/mL to about 1,000 mg/mL, about 400 mg/mL to about 500 mg/mL, about 400 mg/mL to about 600 mg/mL, about 400 mg/mL to about 700 mg/mL, about 400 mg/mL to about 800 mg/mL, about 400 mg/mL to about 900 mg/mL, about 400 mg/mL to about 1,000 mg/mL, about 500 mg/ml to about 600 mg/mL, about 500 mg/mL to about 700 mg/mL, about 500 mg/mL to about 800 mg/mL, about 500 mg/mL to about 900 mg/mL, about 500 mg/mL to about 1,000 mg/mL, about 600 mg/mL to about 700 mg/mL, about 600 mg/mL to about 800 mg/mL, about 600 mg/mL to about 900 mg/mL, or about 600 mg/mL to about 1,000 mg/mL.

Dosing can be single dosage or cumulative (serial dosing), and can be readily determined by one skilled in the art. For instance, treatment of a bacterial infection may comprise a one-time administration of an effective dose of any pharmaceutical composition disclosed herein. Alternatively, treatment of a bacterial infection may comprise multiple administrations of an effective dose of any pharmaceutical composition disclosed herein carried out over a range of frequency or duration, such as, e.g., once daily, twice daily, trice daily, four times daily, six times daily, eight times daily, and/or every 1, 2, 3, 4, 5, 6 or 7 days, or every 1, 2, 3 or 4 weeks. The timing of administration can vary from subject to subject, depending upon such factors as the severity of a subject's symptoms. For example, an effective dose of any pharmaceutical composition disclosed herein can be administered to a subject once or twice daily for an indefinite period of time, or until the subject no longer requires therapy. A person of ordinary skill in the art will recognize that the condition of the subject can be monitored throughout the course of treatment and that the effective amount of any pharmaceutical composition disclosed herein that is administered can be adjusted accordingly.

A therapeutically effective amount of any pharmaceutical composition disclosed herein is capable of reducing the number of bacterial cells or severity of a bacterial infection in a subject suffering from a bacterial infection by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100% as compared to a subject not receiving the same treatment.

A therapeutically effective amount of any pharmaceutical composition disclosed herein is capable of reducing the number of bacterial cells or severity of a bacterial infection in a subject suffering from a bacterial infection by, e.g., about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or at least 100% as compared to a subject not receiving the same treatment.

A therapeutically effective amount of any pharmaceutical composition disclosed herein may be capable of reducing the number of bacterial cells or severity of a bacterial infection in a subject suffering from a bacterial infection by, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70% as compared to a subject not receiving the same treatment.

A therapeutically effective amount of any pharmaceutical composition disclosed herein may have a half-life of about 0.5 hour, about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 1 day, about 2 days, about 3 days, about 4 days, about 5 days, about 6 days, about 7 days, about 1 week, about 2 weeks, about 3 weeks, about 4 weeks, about one month, about two months, about three months, about four months or more.

The period (or duration) of administration of a therapeutically effective amount of any pharmaceutical composition disclosed herein may be for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

A therapeutically effective amount of any pharmaceutical composition disclosed herein may reduce a bacterial infection and/or reduce the bacterial load in a subject by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%.

A therapeutically effective amount of any pharmaceutical composition disclosed herein may reduce a bacterial infection and/or reduce the bacterial load in a subject by, e.g., about 10%, about 15%, about 20%, about 25%, about 30%, about 35%, about 40%, about 45%, about 50%, about 55%, about 60%, about 65%, about 70%, about 75%, about 80%, about 85%, about 90%, about 95% or about 100%.

A therapeutically effective amount of any pharmaceutical composition disclosed may reduce a bacterial infection and/or reduce the bacterial load in a subject by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

Any pharmaceutical composition disclosed herein may reduce the frequency of a symptom of a disorder associated with a bacterial infection incurred over a given time period. Any pharmaceutical composition disclosed herein may reduce the frequency of a symptom of a disorder associated with a bacterial infection incurred over a given time period by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%. In other aspects of this embodiment, any pharmaceutical composition disclosed herein reduces the frequency of a symptom of a disorder associated with a bacterial infection incurred over a given time period by, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

Any of the therapeutic methods of the present specification may include the step of administering the pharmaceutical composition comprising pristinamycin IA or a pharmaceutically acceptable salt thereof and flopristin or a pharmaceutically acceptable salt thereof, or alternatively, a pharmaceutical composition comprising pristinamycin IA or a pharmaceutically acceptable salt thereof and a pharmaceutical composition comprising flopristin or a pharmaceutically acceptable salt (e.g., at the same time or sequentially), at a pharmaceutically effective amount of the two active components combined. The total daily dose should be determined through appropriate medical judgment by a physician and administered once or several times. The specific therapeutically effective dose level for any particular subject may vary depending on various factors well known in the medical art, including the kind and degree of the response to be achieved, pharmaceutical compositions according to whether other agents are used therewith or not, the patient's age, body weight, health condition, gender, and diet, the time and route of administration, the elimination rate of the pharmaceutical composition, the time period of therapy, other drugs used in combination or coincident with any of the pharmaceutical compositions disclosed herein, and like factors well known in the medical arts.

In still another aspect, the present specification provides a use of the therapeutic composition comprising pristinamycin IA or a pharmaceutically acceptable salt thereof and flopristin or a pharmaceutically acceptable salt thereof, or use of a composition comprising pristinamycin IA or a pharmaceutically acceptable salt thereof and a composition comprising flopristin or a pharmaceutically acceptable salt, for the prevention or treatment of a bacterial infection.

In still another aspect, the present specification provides a use of the therapeutic composition comprising flopristin or a pharmaceutically acceptable salt thereof for the prevention or treatment of a bacterial infection.

In one embodiment, the therapeutic dose of any pharmaceutical composition disclosed herein may be administered daily, semi-weekly, weekly, bi-weekly, or monthly. The period of treatment may be for a week, two weeks, a month, two months, four months, six months, eight months, a year, or longer. The initial dose may be larger than a sustaining dose.

In one embodiment, a weekly therapeutic dose of any pharmaceutical composition disclosed herein may be at least 0.05 mg/kg, at least 0.25 mg/kg, at least 0.5 mg/kg, at least 0.75 mg/kg, at least 1 mg/kg, at least 2.5 mg/kg, at least 5 mg/kg, at least 25 mg/kg, at least 50 mg/kg, at least 100 mg/kg, at least 250 mg/kg, or at least 500 mg/kg.

In one embodiment, a weekly therapeutic dose may be at most 1 mg/kg, at most 2.5 mg/kg, at most 5 mg/kg, at most 10 mg/kg, at most 25 mg/kg, at most 50 mg/kg, at most 100 mg/kg, at most 250 mg/kg, at most 500 mg/kg, or at most 700 mg/kg. In a particular aspect, the weekly dose may range from 0.01 mg/kg to 200 mg/kg. In an alternative aspect, the weekly therapeutic dose may range from 1 mg/kg to 100 mg/kg. In an alternative aspect, the weekly dose may range from 5 mg/kg to 75 mg/kg.

The present specification also provides a pharmaceutical composition comprising pristinamycin IA or a pharmaceutically acceptable salt thereof and flopristin or a pharmaceutically acceptable salt thereof, and a pharmaceutical composition comprising flopristin or pharmaceutically acceptable salt thereof, for the administration to a subject. The pharmaceutical compositions disclosed herein may further include a pharmaceutically acceptable carrier, vehicle, excipient, or diluent. As used herein, the term “pharmaceutically acceptable” means that the composition is sufficient to achieve the therapeutic effects without deleterious side effects, and may be readily determined depending on the type of the diseases, the patient's age, body weight, health conditions, gender, and drug sensitivity, administration route, administration mode, administration frequency, duration of treatment, drugs used in combination or coincident with the composition disclosed herein, and other factors known in medicine.

The pharmaceutical compositions disclosed herein may further include a pharmaceutically acceptable carrier. For oral administration, the carrier may include, but is not limited to, a binder, a lubricant, a disintegrant, an excipient, a solubilizer, a dispersing agent, a stabilizer, a suspending agent, a surfactant, an emulsifying agent, a thickening agent, a colorant, and/or a flavorant. For injectable preparations, the carrier may include a buffering agent, a preserving agent, an analgesic, a solubilizer, a co-solvent, an emulsifying agent, an isotonic agent, and/or a stabilizer. For preparations for topical administration, the carrier may include a base, an excipient, a lubricant, a gel, an ointment, a cream, an emulsion, a foam, a spray and/or a preserving agent.

The pharmaceutical compositions disclosed herein may be formulated into a variety of dosage forms in combination with the aforementioned pharmaceutically acceptable carriers. For example, for oral administration, the pharmaceutical composition may be formulated into tablets, troches, capsules (hard or soft), caplets, granules, powders, sachets, sprinkles, lozenges, rapid dissolving strips, elixirs, emulsion, suspensions, syrups or wafers. The pharmaceutical composition may be formulated into suppositories. For injectable preparations, the pharmaceutical composition may be formulated into an ampule as a single dosage form or a multidose container. The pharmaceutical compositions for the injectables may also be formulated into lyophilized powder, solutions, suspensions, emulsion, nanoparticles such as liposomes, micelle dispersion, gel, or long-acting preparations.

On the other hand, examples of the carrier, the excipient, and the diluent suitable for the pharmaceutical formulations include, without limitation, lactose, dextrose, sucrose, sorbitol, mannitol, xylitol, erythritol, maltitol, starch, acacia rubber, alginate, gelatin, calcium phosphate, calcium silicate, cellulose, methylcellulose, microcrystalline cellulose, polyvinylpyrrolidone, water, methylhydroxy-benzoate, propylhydroxybenzoate, talc, magnesium stearate and mineral oils. In addition, the pharmaceutical formulations may further include fillers, anti-coagulating agents, lubricants, humectants, flavorants, and antiseptics.

The pharmaceutical compositions disclosed herein may be formulated into a single dosage form suitable for the patient's body, and preferably is formulated into a preparation according to the typical method in the pharmaceutical field so as to be administered by an oral, parenteral or topical route, including intravenous, intramuscular, intra-arterial, intramedullary, intramedullary, intraventricular, intrathecal, epidural, pulmonary, inhalation, transdermal, subcutaneous, intraperitoneal, intranasal, intracolonic, ocular, intraocular, sublingual, vaginal, or rectal administration, but is not limited thereto.

The pharmaceutical compositions disclosed herein may be used by blending with a variety of pharmaceutically acceptable carriers such as physiological saline or organic solvents or polymers. In order to increase the stability or absorptivity, carbohydrates such as glucose, sucrose or dextrans, surfactants, antioxidants such as ascorbic acid or glutathione, chelating agents, low molecular weight proteins, polymers, or other stabilizers may be used.

The administration dose and frequency of the pharmaceutical compositions disclosed herein are determined by the type of active ingredients, together with various factors such as the disease to be treated, administration route, subject's age, gender, and body weight, and disease severity.

The total effective dose of the pharmaceutical compositions disclosed herein may be administered to a subject in a single dose or may be administered for a long period of time in multiple doses according to a fractionated treatment protocol. In the pharmaceutical compositions disclosed herein, the content and amount of active ingredients may vary depending on the disease severity. Preferably, the total effective or therapeutic daily dose of any pharmaceutical composition disclosed herein may be about 0.01 μg to about 100 mg per 1 kg of body weight of a subject. However, the effective dose of the antibiotic is determined considering various factors including subject's age, body weight, health conditions, gender, disease severity, diet, other co-administered drugs, in addition to administration route and treatment frequency of the pharmaceutical composition. In view of this, those skilled in the art may easily determine an effective dose suitable for the particular use of the pharmaceutical composition disclosed herein. The pharmaceutical composition disclosed herein is not particularly limited to the formulation, and administration route and mode, as long as it shows suitable effects.

Moreover, the pharmaceutical compositions disclosed herein may be administered in combination or concurrently with other pharmaceutical formulations with or within an active agent showing prophylactic or therapeutic efficacy.

Given the teachings and guidance provided herein, those skilled in the art will understand that a formulation described herein can be equally applicable to many types of antibiotics and other therapeutic compounds, including those exemplified, as well as others known in the art. Given the teachings and guidance provided herein, those skilled in the art also will understand that the selection of, for example, type(s) or and/or amount(s) of one or more excipients, surfactants and/or optional components can be made based on the chemical and functional compatibility with the biopharmaceutical to be formulated and/or the mode of administration as well as other chemical, functional, physiological and/or medical factors well known in the art.

In various embodiments, the pharmaceutical compositions disclosed herein also can include, without limitation, two or more different therapeutic compounds for a single or multiple conditions. Use of multiple therapeutic compounds in a formulation can be directed to, for example, the same or different indications. Similarly, in another embodiment, multiple therapeutic compounds can be used in a formulation to treat, for example, both a pathological condition and one or more side effects caused by the primary treatment. In a further embodiment, multiple therapeutic compounds also can be included, without limitation, in a pharmaceutical composition as described herein to accomplish different medical purposes including, for example, simultaneous treatment and monitoring of the progression of the pathological condition. In an additional embodiment, multiple, concurrent therapies such as those exemplified herein as well as other combinations well known in the art are particularly useful for patient compliance because a single pharmaceutical composition can be sufficient for some or all suggested treatments and/or diagnoses. Those skilled in the art will know those therapeutic compounds that can be admixed for a wide range of combination therapies. Similarly, in various embodiments, a first therapeutic compound can be used with a second or more therapeutic compound and combinations of one or more therapeutic compounds together with one or more other therapeutic compounds, including a small molecule (e.g., another antibiotic) or an antibody pharmaceutical. Therefore, in various embodiments a formulation is provided containing 1, 2, 3, 4, 5 or 6 or more different therapeutic compounds, as well as, for one or more therapeutic compounds combined with one or more other therapeutic compounds.

In various embodiments, the pharmaceutical compositions disclosed herein can include, one or more preservatives and/or additives known in the art. Similarly, a pharmaceutical composition can further be formulated, without limitation, into any of various known delivery formulations. For example, in an embodiment, a pharmaceutical composition can include, surfactants, adjuvant, biodegradable polymers, hydrogels, etc., such optional components, their chemical and functional characteristics are known in the art. Similarly known in the art are pharmaceutical compositions that facilitate rapid, sustained or delayed release of the bioactive agents after administration. A formulation as described can be produced to include these or other formulation components known in the art.

In any of the methods or compositions described herein, the ratio of pristinamycin IA to flopristin (PIA:flopristin) by weight may be about 1:99, about 2:98, about 3:97, about 4:96, about 5:95, about 6:94, about 7:93, about 8:92, about 9:91, about 10:90, about 11:89, about 12:88, about 13:87, about 14:86, about 15:85, about 16:84, about 17:83, about 18:82, about 19:81, about 20:80, about 21:79, about 22:78, about 23:77, about 24:76, about 25:75, about 26:74, about 27:73, about 28:72, about 29:71, about 30:70, about 31:69, about 32:68, about 33:67, about 34:66, about 35:65, about 36:64, about 37:63, about 38:62, about 39:61, about 40:60, about 41:59, about 42:58, about 43:57, about 44:56, about 45:55, about 46:54, about 47:53, about 48:52, about 49:51, about 50:50, about 51:49, about 52:48, about 53:47, about 54:46, about 55:45, about 56:44, about 57:43, about 58:42, about 59:41, about 60:40, about 61:39, about 62:38, about 63:37, about 64:36, about 65:35, about 66:34, about 67:33, about 68:32, about 69:31, about 70:30, about 71:29, about 72:28, about 73:27, about 74:26, about 75:25, about 76:24, about 77:23, about 78:22, about 79:21, about 80:20, about 81:19, about 82:18, about 83:17, about 84:16, about 85:15, about 86:14, about 87:13, about 88:12, about 89:11, about 90:10, about 91:9, about 92:8, about 93:7, about 94:6, about 95:5, about 96:4, about 97:3, about 98:2, or about 99:1.

The term “pharmaceutically acceptable salts” is meant to include salts of the active compounds that are prepared with relatively nontoxic acids or bases, depending on the particular substituents found on the compounds described herein. When compounds disclosed herein contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogen-phosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, ptolylsulfonic, citric, tartaric, oxalic, methanesulfonic, and the like. Also included are salts of amino acids such as arginate and the like, and salts of organic acids like glucuronic or galactunoric acids and the like (e.g., Berge et al., “Pharmaceutical Salts”, Journal of Pharmaceutical Science, 1977, 66, 1-19). Certain specific compounds disclosed herein contain both basic and acidic functionalities that allow the compounds to be converted into either base or acid addition salts.

Certain compounds disclosed herein can exist in unsolvated forms and solvated forms, including hydrated forms. In general, the solvated forms are equivalent to unsolvated forms and are encompassed within the scope of the present disclosure. Certain compounds disclosed herein may exist in multiple crystalline or amorphous forms. In general, all physical forms are equivalent for the uses contemplated by the present disclosure and are intended to be within the scope of the present disclosure.

### Pharmaceutical Composition

In an embodiment, a pharmaceutical composition comprising pristinamycin IA or a pharmaceutically acceptable salt thereof and flopristin or a pharmaceutically acceptable salt thereof wherein the ratio of pristinamycin IA to flopristin (PIA:flopristin) by weight is about 1:99, about 2:98, about 3:97, about 4:96, about 5:95, about 6:94, about 7:93, about 8:92, about 9:91, about 10:90, about 11:89, about 12:88, about 13:87, about 14:86, about 15:85, about 16:84, about 17:83, about 18:82, about 19:81, about 20:80, about 21:79, about 22:78, about 23:77, about 24:76, about 25:75, about 26:74, about 27:73, about 28:72, about 29:71, about 30:70, about 31:69, about 32:68, about 33:67, about 34:66, about 35:65, about 36:64, about 37:63, about 38:62, about 39:61, about 40:60, about 41:59, about 42:58, about 43:57, about 44:56, about 45:55, about 46:54, about 47:53, about 48:52, about 49:51, about 50:50, about 51:49, about 52:48, about 53:47, about 54:46, about 55:45, about 56:44, about 57:43, about 58:42, about 59:41, about 60:40, about 61:39, about 62:38, about 63:37, about 64:36, about 65:35, about 66:34, about 67:33, about 68:32, about 69:31, about 70:30, about 71:29, about 72:28, about 73:27, about 74:26, about 75:25, about 76:24, about 77:23, about 78:22, about 79:21, about 80:20, about 81:19, about 82:18, about 83:17, about 84:16, about 85:15, about 86:14, about 87:13, about 88:12, about 89:11, about 90:10, about 91:9, about 92:8, about 93:7, about 94:6, about 95:5, about 96:4, about 97:3, about 98:2, or about 99:1.

In another embodiment is provided a kit comprising a first composition containing pristinamycin IA or a pharmaceutically acceptable salt thereof and a second composition containing flopristin or a pharmaceutically acceptable salt thereof, wherein the pristinamycin IA:flopristin ratio by weight is about 1:99, about 2:98, about 3:97, about 4:96, about 5:95, about 6:94, about 7:93, about 8:92, about 9:91, about 10:90, about 11:89, about 12:88, about 13:87, about 14:86, about 15:85, about 16:84, about 17:83, about 18:82, about 19:81, about 20:80, about 21:79, about 22:78, about 23:77, about 24:76, about 25:75, about 26:74, about 27:73, about 28:72, about 29:71, about 30:70, about 31:69, about 32:68, about 33:67, about 34:66, about 35:65, about 36:64, about 37:63, about 38:62, about 39:61, about 40:60, about 41:59, about 42:58, about 43:57, about 44:56, about 45:55, about 46:54, about 47:53, about 48:52, about 49:51, about 50:50, about 51:49, about 52:48, about 53:47, about 54:46, about 55:45, about 56:44, about 57:43, about 58:42, about 59:41, about 60:40, about 61:39, about 62:38, about 63:37, about 64:36, about 65:35, about 66:34, about 67:33, about 68:32, about 69:31, about 70:30, about 71:29, about 72:28, about 73:27, about 74:26, about 75:25, about 76:24, about 77:23, about 78:22, about 79:21, about 80:20, about 81:19, about 82:18, about 83:17, about 84:16, about 85:15, about 86:14, about 87:13, about 88:12, about 89:11, about 90:10, about 91:9, about 92:8, about 93:7, about 94:6, about 95:5, about 96:4, about 97:3, about 98:2, or about 99:1, wherein each composition further includes a pharmaceutically acceptable carrier or excipients, wherein the kit is optionally packaged as a blister pack, and wherein the kit includes instructions to administer to a subject in need thereof the first composition and the second composition at the same time, or to administer the first composition before or after the second composition is administered.

“Pharmaceutically acceptable excipient” and “pharmaceutically acceptable carrier” refer to a substance that aids the administration of an active agent to and absorption by a subject or can facilitate the distribution or delivery of the active ingredients to the site of action and can be included in the compositions disclosed herein without causing a significant adverse toxicological effect on the subject. Non-limiting examples of pharmaceutically acceptable excipients include water, NaCl, normal saline solutions, lactated Ringer's, normal sucrose, dextrose, normal glucose, binders, fillers, disintegrants, lubricants, coatings, sweeteners, flavors, salt solutions (such as Ringer's solution), alcohols, oils, lipids, gelatins, carbohydrates such as lactose, amylase or starch, fatty acid esters, hydroxymethylcellulose, polyvinyl pyrrolidine, polymers and colors, and the like. Such preparations can be sterilized and, if desired, mixed with auxiliary agents such as lubricants, preservatives, stabilizers, wetting agents, emulsifiers, surfactants, salts for influencing osmotic pressure, buffers, coloring, and/or aromatic substances and the like that do not deleteriously react with the compounds of the disclosure. One of skill in the art will recognize that other pharmaceutical excipients are useful in the present disclosure.

Pharmaceutical formulations for parenteral administration, e.g., by bolus injection or continuous infusion, include aqueous solutions of the active compounds in water soluble form. Additionally, suspensions or emulsions of the active compounds may be prepared as appropriate oily injectable nanosuspensions or nanoemulsions. Suitable lipophilic solvents or vehicles include fatty oils such as sesame oil, or other organic oils such as soybean, grapefruit or almond oils, or synthetic fatty acid esters, such as ethyl oleate or triglycerides, or phospholipids or other lipids. Aqueous injection suspensions may contain substances which increase the viscosity of the suspension, such as sodium carboxymethyl cellulose, sorbitol, or dextran. Optionally, the suspension may also contain suitable stabilizers, surfactants or agents that increase the solubility of the compounds to allow for the preparation of highly concentrated solutions, suspensions, emulsions or semisolids such as gels. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use. The active ingredients may also be complexed with proteins (such as albumin), polymers or be formulated into hydrophilic gels or into liposomes or other nanoparticle formulations for injection.

For topical administration, pharmaceutical compositions may be formulated as is known in the art for direct application to a target area. Forms chiefly conditioned for topical application take the form, for example, of creams, milks, gels, powders, dispersion or microemulsions, eye drops, ear drops, nose drops, lotions thickened to a greater or lesser extent, impregnated pads, transdermal patches, ointments or sticks, aerosol formulations (e.g. sprays or foams), soaps, detergents, lotions or cakes of soap. The pharmaceutical composition may further be formulated for topical administration in the mouth or throat. For example, the active ingredients may be formulated as a lozenge further comprising a flavoured base, usually sucrose and acacia or tragacanth; pastilles comprising the composition in an inert base such as gelatin and glycerin or sucrose and acacia; and mouthwashes comprising the composition of the present invention in a suitable liquid carrier.

For oral administration, the compounds can be formulated readily by combining the active compounds with pharmaceutically acceptable carriers well known in the art. Such carriers enable the compounds of the invention to be formulated as tablets, pills, dragees, capsules, caplets, liquids, gels, films, syrups, elixirs, slurries, sachets, powder, sprinkle, pellets, rapid dissolving strips, suspensions, emulsions and the like, for oral ingestion by a patient to be treated. Pharmaceutical preparations for oral use can be obtained by combining the active compounds with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth methyl cellulose, hydroxypropylmethyl cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Additionally, the pharmaceutical composition may be formulated as sustained release dosage forms and the like. The formulations can be so constituted that they release the active agents, for example, in a particular part of the intestinal or respiratory tract, possibly over a period of time. Coatings, envelopes, and protective matrices may be made, for example, from polymeric substances, such as polylactideglycolates, liposomes, microemulsions, microparticles, nanoparticles, or waxes. These coatings, envelopes, and protective matrices are useful to coat indwelling devices, e.g., stents, catheters, peritoneal dialysis tubing, draining devices and the like.

The pharmaceutical compositions of the present invention can also be administered to the respiratory tract. For administration by inhalation or insufflation, the composition may take the form of a nebulizing liquid or dry powder, for example, a powder mix of the therapeutic agent and a suitable powder base such as lactose or starch. Pharmaceutical compositions of the present invention can also be administered in an aqueous solution or nanoparticle suspensions when administered in an aerosol or inhaled form. Thus, other aerosol pharmaceutical formulations may comprise, for example, a physiologically acceptable buffered saline solution containing e.g., about 0.01 mg/mL to about 1,000 mg/mL, about 0.1 mg/mL to about 1,000 mg/mL, about 1 mg/mL to about 1,000 mg/mL, about 10 mg/mL to about 1,000 mg/mL, about 25 mg/mL to about 1,000 mg/mL, about 50 mg/mL to about 1000 mg/mL, about 100 mg/mL to about 1,000 mg/mL, about 200 mg/mL to about 1,000 mg/mL, about 500 mg/mL to about 1,000 mg/mL, about 750 mg/mL to about 1,000 mg/mL, about 1 mg/mL to about 10 mg/mL, about 1 mg/mL to about 50 mg/mL, about 1 mg/mL to about 100 mg/mL, about 1 mg/mL to about 200 mg/mL, about 1 mg/mL to about 300 mg/mL, about 1 mg/mL to about 400 mg/mL, about 1 mg/mL to about 500 mg/mL, about 1 mg/mL to about 600 mg/mL, about 1 mg/mL to about 700 mg/mL, about 1 mg/mL to about 800 mg/mL, about 1 mg/mL to about 900 mg/mL, about 1 mg/mL to about 1,000 mg/mL, about 10 mg/mL to about 50 mg/mL, about 10 mg/mL to about 100 mg/mL, about 10 mg/mL to about 200 mg/mL, about 10 mg/mL to about 300 mg/mL, about 10 mg/mL to about 400 mg/mL, about 10 mg/mL to about 500 mg/mL, about 10 mg/mL to about 600 mg/mL, about 10 mg/mL to about 700 mg/mL, about 10 mg/mL to about 800 mg/mL, about 10 mg/mL to about 900 mg/mL, about 10 mg/mL to about 1,000 mg/mL, about 50 mg/mL to about 100 mg/mL, about 50 mg/mL to about 200 mg/mL, about 50 mg/mL to about 300 mg/mL, about 50 mg/mL to about 400 mg/mL, about 50 mg/mL to about 500 mg/mL, about 50 mg/mL to about 600 mg/mL, about 50 mg/mL to about 700 mg/mL, about 50 mg/mL to about 800 mg/mL, about 50 mg/mL to about 900 mg/mL, about 50 mg/mL to about 1,000 mg/mL, about 100 mg/mL to about 200 mg/mL, about 100 mg/mL to about 300 mg/mL, about 100 mg/mL to about 400 mg/mL, about 100 mg/mL to about 500 mg/mL, about 100 mg/mL to about 600 mg/mL, about 100 mg/mL to about 700 mg/mL, about 100 mg/mL to about 800 mg/mL, about 100 mg/mL to about 900 mg/mL, about 100 mg/mL to about 1,000 mg/mL, about 200 mg/mL to about 300 mg/mL, about 200 mg/mL to about 400 mg/mL, about 200 mg/mL to about 500 mg/mL, about 200 mg/mL to about 600 mg/mL, about 200 mg/mL to about 700 mg/mL, about 200 mg/mL to about 800 mg/mL, about 200 mg/mL to about 900 mg/mL, about 200 mg/mL to about 1,000 mg/mL, about 300 mg/mL to about 400 mg/mL, about 300 mg/mL to about 500 mg/mL, about 300 mg/mL to about 600 mg/mL, about 300 mg/mL to about 700 mg/mL, about 300 mg/mL to about 800 mg/mL, about 300 mg/mL to about 900 mg/mL, about 300 mg/mL to about 1,000 mg/mL, about 400 mg/mL to about 500 mg/mL, about 400 mg/mL to about 600 mg/mL, about 400 mg/mL to about 700 mg/mL, about 400 mg/mL to about 800 mg/mL, about 400 mg/mL to about 900 mg/mL, about 400 mg/mL to about 1,000 mg/mL, about 500 mg/mL to about 600 mg/mL, about 500 mg/mL to about 700 mg/mL, about 500 mg/mL to about 800 mg/mL, about 500 mg/mL to about 900 mg/mL, about 500 mg/mL to about 1,000 mg/mL, about 600 mg/mL to about 700 mg/mL, about 600 mg/mL to about 800 mg/mL, about 600 mg/mL to about 900 mg/mL, or about 600 mg/mL to about 1,000 mg/ml of the active agents of the present invention specific for the indication or disease to be treated.

Drops, such as eye drops, ear drops, or nose drops, may be formulated with the active agents of the present invention in an aqueous or non-aqueous base also comprising one or more dispersing agents, solubilizing agents or suspending agents. Liquid sprays can be pumped, or are conveniently delivered from pressurized packs. Drops can be delivered via a simple eye dropper-capped bottle, via a plastic bottle adapted to deliver liquid contents drop-wise, or via a specially shaped closure.

Also provided herein are combinatorial methods for developing suitable pharmaceutical compositions using combinations of amino acids as an excipient. These methods are effective for developing stable liquid or lyophilized pharmaceutical compositions, and particularly pharmaceutical compositions that comprise one or more therapeutic compounds.

Compositions in accordance with embodiments described herein have desirable properties, such as desirable solubility, viscosity, syringeability and stability. Lyophilates in accordance with embodiments described herein have desirable properties, such as desirable recovery, stability and reconstitution.

In an embodiment, the pH of any pharmaceutical composition disclosed herein is at least about 1, 1.5, 2, 2.5, 3, 3.5, 3.75, 4, 4.25, 4.5, 4.75, 5, 5.25, 5.5, 5.75, 6, 6.25, 6.5, 6.75, 7, 7.25, 7.5, 7.75, 8, 8.25, 8.5, 8.75, 9, 9.25, 9.5, 9.75, 10, 10.25, 10.5, 10.75, 11, 11.25, 11.5, 11.75 or 12.

In an embodiment, the pH of any pharmaceutical composition disclosed herein is from about 3 to about 9, about 3 to about 8, about 3 to about 7, about 3 to about 6, about 3 to about 5, about 3 to about 4, about 4 to about 9, about 4 to about 8, about 4 to about 7, about 4 to about 6, about 4 to about 5, about 5 to about 9, about 5 to about 8, about 5 to about 7, about 5 to about 6, about 6 to about 9, about 6 to about 8, about 6 to about 7, about 7 to about 9, about 7 to about 8, or about 8 to about 9.

### Method of Treatment and Prevention

As a general proposition, a therapeutically effective amount or prophylactically effective amount of a streptogramin B (e.g., pristinamycin IA or a pharmaceutically acceptable salt thereof) and a streptogramin A (e.g., flopristin or a pharmaceutically acceptable salt thereof) either together within a single pharmaceutical composition (dosage form) or separately in two independent pharmaceutical compositions (dosage forms), e.g., in a kit, can be used in methods for treating or preventing a bacterial infection or a condition caused by a bacterial infection. Any of the pharmaceutical compositions or kits described herein can be used in any of the methods.

In any of the therapeutic methods disclosed herein, the pharmaceutical composition comprising pristinamycin IA or a pharmaceutically acceptable salt thereof and flopristin or a pharmaceutically acceptable salt thereof, wherein the pristinamycin IA (PIA) to flopristin (PIA:flopristin) ratio by weight is about 1:99, about 2:98, about 3:97, about 4:96, about 5:95, about 6:94, about 7:93, about 8:92, about 9:91, about 10:90, about 11:89, about 12:88, about 13:87, about 14:86, about 15:85, about 16:84, about 17:83, about 18:82, about 19:81, about 20:80, about 21:79, about 22:78, about 23:77, about 24:76, about 25:75, about 26:74, about 27:73, about 28:72, about 29:71, about 30:70, about 31:69, about 32:68, about 33:67, about 34:66, about 35:65, about 36:64, about 37:63, about 38:62, about 39:61, about 40:60, about 41:59, about 42:58, about 43:57, about 44:56, about 45:55, about 46:54, about 47:53, about 48:52, about 49:51, about 50:50, about 51:49, about 52:48, about 53:47, about 54:46, about 55:45, about 56:44, about 57:43, about 58:42, about 59:41, about 60:40, about 61:39, about 62:38, about 63:37, about 64:36, about 65:35, about 66:34, about 67:33, about 68:32, about 69:31, about 70:30, about 71:29, about 72:28, about 73:27, about 74:26, about 75:25, about 76:24, about 77:23, about 78:22, about 79:21, about 80:20, about 81:19, about 82:18, about 83:17, about 84:16, about 85:15, about 86:14, about 87:13, about 88:12, about 89:11, about 90:10, about 91:9, about 92:8, about 93:7, about 94:6, about 95:5, about 96:4, about 97:3, about 98:2, or about 99:1.

In any of the therapeutic methods disclosed herein, a kit comprising a first pharmaceutical composition containing pristinamycin IA or a pharmaceutically acceptable salt thereof and a second pharmaceutical composition containing flopristin or a pharmaceutically acceptable salt thereof, wherein the pristinamycin IA:flopristin ratio by weight is about 1:99, about 2:98, about 3:97, about 4:96, about 5:95, about 6:94, about 7:93, about 8:92, about 9:91, about 10:90, about 11:89, about 12:88, about 13:87, about 14:86, about 15:85, about 16:84, about 17:83, about 18:82, about 19:81, about 20:80, about 21:79, about 22:78, about 23:77, about 24:76, about 25:75, about 26:74, about 27:73, about 28:72, about 29:71, about 30:70, about 31:69, about 32:68, about 33:67, about 34:66, about 35:65, about 36:64, about 37:63, about 38:62, about 39:61, about 40:60, about 41:59, about 42:58, about 43:57, about 44:56, about 45:55, about 46:54, about 47:53, about 48:52, about 49:51, about 50:50, about 51:49, about 52:48, about 53:47, about 54:46, about 55:45, about 56:44, about 57:43, about 58:42, about 59:41, about 60:40, about 61:39, about 62:38, about 63:37, about 64:36, about 65:35, about 66:34, about 67:33, about 68:32, about 69:31, about 70:30, about 71:29, about 72:28, about 73:27, about 74:26, about 75:25, about 76:24, about 77:23, about 78:22, about 79:21, about 80:20, about 81:19, about 82:18, about 83:17, about 84:16, about 85:15, about 86:14, about 87:13, about 88:12, about 89:11, about 90:10, about 91:9, about 92:8, about 93:7, about 94:6, about 95:5, about 96:4, about 97:3, about 98:2, or about 99:1.

In one embodiment is provided a method of treating or preventing a bacterial infection in a subject in need thereof comprising administering to said subject a therapeutically effective amount of a pharmaceutical composition comprising pristinamycin IA or a pharmaceutically acceptable salt thereof and flopristin or a pharmaceutically acceptable salt thereof, wherein the pristinamycin IA:flopristin ratio by weight ranges from 1:99 to 99:1, wherein the bacterial infection is caused by one or more susceptible and/or resistant strains of gram-positive bacteria, gram-negative bacteria, atypical bacteria, aerobic bacteria, and/or anaerobic bacteria, such as Bacillus spp., Bacteroides spp., Bordetella spp., Borrelia spp., Brucella spp., Burkholderia spp., Campylobacter spp., Chlamydia spp., Clostridium spp., Corynebacterium spp., Coxiella spp., Ehrlichia spp., Enterococcus spp., Francisella spp., Fusobacterium spp., Gardnerella spp., Haemophilus spp., Kingella spp., Legionella spp., Listeria spp., Moraxella spp., Mycoplasma spp., Mycobacterium spp., Neisseria spp., Nocardia spp., Peptostreptococcus spp., Porphyromonas spp., Propionibacterium spp., Prevotella spp., Rickettsia spp., Salmonella spp., Shigella spp., Staphylococcus spp., Streptococcus spp., Ureaplasma spp., Vibrio spp., or Yersinia spp.

A method of treating or preventing a respiratory infection and/or disease in a subject in need thereof may comprise administering to said subject a therapeutically effective amount of a pharmaceutical composition comprising pristinamycin IA or a pharmaceutically acceptable salt thereof and flopristin or a pharmaceutically acceptable salt thereof, wherein the pristinamycin IA:flopristin ratio by weight ranges from 1:99 to 99:1. The respiratory infections and/or diseases may comprise community acquired pneumonia or healthcare associated pneumonia, chronic bronchitis, sinusitis, acute maxillary sinusitis, acute exacerbations of chronic bronchitis, pharyngitis, chronic obstructive pulmonary disease, and non-tuberculosis mycobacteria infection caused by or associated with bacteria, such as one or more strains of susceptible and/or resistant Streptococcus pneumoniae, Streptococcus pyogenes, Staphylococcus aureus, Haemophilus influenzae, Haemophilus parainfluenzae, Moraxella catarrhalis, Legionella spp., Chlamydia pneumoniae, Mycoplasma pneumoniae, Bordetella pertussis, Neisseria gonorrhoeae, Corynebacterium diphtheriae, Coxiella burnettii, Peptostreptococcus spp., Fusobacterium spp., Bacteroides spp., Prevotella spp., Nocardia spp., or Mycobacterium spp.

A method of treating or preventing a sexually transmitted infection or a genitourinary tract infection in a subject in need thereof may comprise administering to said subject a therapeutically effective amount of a pharmaceutical composition comprising pristinamycin IA or a pharmaceutically acceptable salt thereof and flopristin or a pharmaceutically acceptable salt thereof, wherein the pristinamycin IA:flopristin ratio by weight ranges from 1:99 to 99:1. The sexually transmitted infection or genitourinary tract infection may be caused by or associated with bacteria, such as by one or more strains of susceptible and/or resistant Neisseria gonorrhoeae, Chlamydia trachomatis, Mycoplasma genitalium, Mycoplasma hominis, Ureaplasma urealyticum, Ureaplasma parvum, or Gardnerella vaginalis.

A method of treating or preventing skin and/or soft tissue infections and/or disease in a subject in need thereof may comprise administering to said subject a therapeutically effective amount of a pharmaceutical composition comprising pristinamycin IA or a pharmaceutically acceptable salt thereof and flopristin or a pharmaceutically acceptable salt thereof, wherein the pristinamycin IA:flopristin ratio by weight ranges from 1:99 to 99:1. The skin and/or soft tissue infections and/or disease may be caused by bacteria, such as by one or more strains of susceptible and/or resistant Staphylococcus aureus, Staphylococcus haemolyticus, Staphylococcus lugdunensis, Streptococcus agalactiae, viridans group streptococci, Streptococcus pyogenes, Enterococcus faecalis, Enterococcus faecium, Corynebacterium spp., or Propionibacterium acnes.

A method of treating or preventing an infection in subjects with cystic fibrosis in need thereof may comprise administering to said subject a therapeutically effective amount of pristinamycin IA or a pharmaceutically acceptable salt thereof and flopristin or a pharmaceutically acceptable salt thereof, wherein the pristinamycin IA:flopristin ratio by weight ranges from 1:99 to 99:1. The infection or threat of infection may be caused by or associated with bacteria, such as one or more strains of susceptible and/or resistant Staphylococcus aureus, Haemophilus influenzae, or Mycobacterium spp.

A method of treating or preventing a bone and/or joint infection in a subject in need thereof may comprise administering to said subject a therapeutically effective amount of a pharmaceutical composition comprising pristinamycin IA or a pharmaceutically acceptable salt thereof and flopristin or a pharmaceutically acceptable salt thereof, wherein the pristinamycin IA:flopristin ratio by weight ranges from 1:99 to 99:1. The infection or threat of infection may be caused by bacteria, such as one or more strains of susceptible and/or resistant Staphylococcus aureus, Staphylococcus haemolyticus, Staphylococcus lugdunensis, Streptococcus agalactiae, Streptococcus pyogenes, Streptococcus pneumoniae, viridans group streptococci, Salmonella spp., Shigella spp., Campylobacter spp., Yersinia spp., Enterococcus faecalis, Enterococcus faecium, Kingella kingae, Corynebacterium spp., Propionibacterium acnes, Chlamydia trachomatis or Neisseria gonorrhoeae.

A method of treating or preventing endocarditis in a subject in need thereof comprising administering to said subject a therapeutically effective amount of a pharmaceutical composition comprising pristinamycin IA or a pharmaceutically acceptable salt thereof and flopristin or a pharmaceutically acceptable salt thereof, wherein the pristinamycin IA:flopristin ratio by weight ranges from 1:99 to 99:1. The infection or threat of infection may be caused by bacteria, such as one or more strains of susceptible and/or resistant Staphylococcus aureus, Staphylococcus haemolyticus, Staphylococcus lugdunensis, Streptococcus gallolyticus, viridans group streptococci, Enterococcus faecalis, Enterococcus faecium, Haemophilus spp., Kingella spp., Legionella spp., or Corynebacterium spp.

A method of treating or preventing bacteremia or sepsis in a subject in need thereof comprising administering to said subject a therapeutically effective amount of a pharmaceutical composition comprising pristinamycin IA or a pharmaceutically acceptable salt thereof and flopristin or a pharmaceutically acceptable salt thereof, wherein the pristinamycin IA:flopristin ratio by weight ranges from 1:99 to 99:1. The infection or threat of infection may be caused by one or more strains of susceptible and/or resistant bacteria.

A method of treating or preventing anthrax, tularemia, plague, glanders, or meliodosis in a subject in need thereof comprising administering to said subject a therapeutically effective amount of a pharmaceutical composition comprising pristinamycin IA or a pharmaceutically acceptable salt thereof and flopristin or a pharmaceutically acceptable salt thereof, wherein the pristinamycin IA:flopristin ratio by weight ranges from 1:99 to 99:1. The infection or threat of infection may be caused by bacteria, such as one or more strains of susceptible and/or resistant Bacillus anthracis, Francisella tularensis, Yersinia pestis, Burkholderia mallei, or Burkholderia pseudomallei.

A method of treating or preventing a bacterial infection in a subject in need thereof comprising administering to said subject a therapeutically effective amount of a pharmaceutical composition comprising flopristin or a pharmaceutically acceptable salt thereof. The infection or threat of infection may be caused by caused by one or more susceptible and/or resistant strains of gram-positive bacteria, gram-negative bacteria, atypical bacteria, aerobic bacteria, and/or anaerobic bacteria.

A method of treating or preventing a sexually transmitted infection or a genitourinary tract infection in a subject in need thereof comprising administering to said subject a therapeutically effective amount of a pharmaceutical composition comprising flopristin or a pharmaceutically acceptable salt thereof. The sexually transmitted infection or genitourinary tract infection may be caused by or associated with bacteria, such as by one or more strains of susceptible and/or resistant Neisseria gonorrhoeae, Chlamydia trachomatis, Mycoplasma genitalium, Mycoplasma hominis, Ureaplasma urealyticum, Ureaplasma parvum, or Gardnerella vaginalis.

In any of the methods for treating or preventing a bacterial infection, the subject can be a human or an animal.

In any of the methods of treating or preventing bacterial infections, the route of administration can be oral, rectal, transmucosal, inhalation, intranasal, topical (including dermal, ophthalmic, otic), vaginal, intestinal, buccal, or sublingual administration; parenteral delivery, including intramuscular, subcutaneous, intravenous, or intramedullary injections, as well as intrathecal, epidural, intraventricular, intraperitoneal, intraamniotic, intranasal, or intraocular injections or injection into the joints.

“Treating” and “treatment” methods include administering to a subject a therapeutically effective amount of an active agent. The administering step may consist of a single administration or may include a series of administrations. The length of the treatment period depends on a variety of factors, such as the severity of the condition, the age of the patient, the concentration of active agent, the activity of the compositions used in the treatment, or a combination thereof. It will also be appreciated that the effective dosage of an agent used for the treatment may increase or decrease over the course of a particular treatment regime. Changes in dosage may result and become apparent by standard diagnostic assays known in the art. In some instances, chronic administration may be required.

Preventive or prophylactic treatment methods include administering to a subject a therapeutically effective amount of an active agent or a combination of active agents. The administering step may consist of a single administration or may include a series of administrations. The length of the treatment period depends on a variety of factors, such as the severity of the condition, the age of the subject, the concentration of active agent, the activity of the compositions used in the treatment, or a combination thereof. It will also be appreciated that the effective dosage of an agent used for the preventive or prophylaxis may increase or decrease over the course of a particular preventive or prophylaxis treatment regime. Changes in dosage may result and become apparent by standard diagnostic assays known in the art. In some instances, chronic administration may be required.

An “effective amount” is an amount sufficient for a compound to accomplish a stated purpose relative to the absence of the compound (e.g. achieve the effect for which it is administered, treat a disease, reduce enzyme activity, increase enzyme activity, reduce binding to a target or receptor, reduce or block a signaling pathway, or reduce one or more symptoms of a disease or condition). An example of an “effective amount” is an amount sufficient to contribute to the treatment, prevention, or reduction of bacterial load, or symptoms of a disease, which could also be referred to as a “therapeutically effective amount”.

The therapeutically effective amount can be initially determined from cell culture assays or pharmacokinetic studies. Target concentrations will be those concentrations of active compound(s) that are capable of achieving the methods of treatment of prevention described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and/or plasma concentrations and adjusting the dosage upwards or downwards, as described above. Adjusting the dose to achieve maximal efficacy in humans based on the methods described above and other methods is well within the capabilities of the ordinarily skilled artisan.

Dosages may be varied depending upon the requirements of the subject and the compound being employed. The dose administered to a subject, in the context of the present disclosure should be sufficient to affect a beneficial therapeutic response in the patient over time. The size of the dose also will be determined by the existence, nature, and extent of any adverse side-effects. Determination of the proper dosage for a particular situation is within the skill of the practitioner. Treatment may be initiated with smaller dosages which are less than the optimum dose of the compound. Thereafter, the dosage may be increased by small increments until the optimum effect under circumstances is reached. Dosage amounts and intervals can be adjusted individually to provide levels of the administered compound effective for the clinical indication being treated. This will provide a therapeutic regimen that is commensurate with the severity of the subject's disease state.

### Route of Administration

As a general proposition, a therapeutically effective amount or prophylactically effective amount of a streptogramin B (e.g., pristinamycin IA or a pharmaceutically acceptable salt thereof) and a streptogramin A (e.g., flopristin or a pharmaceutically acceptable salt thereof) either together within a single pharmaceutical composition (dosage form) or separately in two independent pharmaceutical compositions (dosage forms), e.g., in a kit, can be administered by routes of administration described herein. Any of the pharmaceutical compositions or kits described herein can be administered in any of the routes of administration.

The pharmaceutical composition comprising pristinamycin IA or a pharmaceutically acceptable salt thereof and flopristin or a pharmaceutically acceptable salt thereof, or the pharmaceutical composition comprising flopristin or a pharmaceutically acceptable salt thereof can therefore be administered as a single dose, or as two or more doses (which may or may not contain the same amount of the desired molecule) over time, intravenously, or as a continuous infusion via an implantation device or catheter. Further refinement of the appropriate dosage is routinely made by those of ordinary skill in the art and is within the ambit of tasks routinely performed by them. Appropriate dosages may be ascertained through use of appropriate dose-response data. In various embodiments, the therapeutic compounds in a pharmaceutical composition described herein can, without limitation, be administered to patients throughout an extended time period, such as chronic administration for a chronic condition. The composition can be a solid, a semi-solid, a liquid, a suspension, or an aerosol; optionally, the composition, first composition, or second composition is a powder, granule, tablet, geltab, lozenge, rapid dissolving strip, capsule (hard or soft), caplet, syrup, elixir, suspension, emulsion, sachet, sprinkle, pellet, patch, spray, cream, ointment, gel, lotion or foam.

In an embodiment, for oral, rectal, vaginal, parenteral, pulmonary, sublingual and/or intranasal delivery formulations, tablets can be made by compression or molding, optionally with one or more accessory ingredients or additives. In an embodiment, compressed tablets are prepared, for example, by compressing in a suitable tabletting machine, the therapeutic compounds in a free-flowing form such as a powder or granules, optionally mixed with a binder (for example, without limitation, povidone, gelatin, hydroxypropylmethyl cellulose), lubricant, inert diluent, preservative, surfactants, disintegrant (for example, without limitation, sodium starch glycolate, cross-linked povidone, cross-linked sodium carboxymethyl cellulose) and/or surface-active or dispersing agent.

In an embodiment, molded tablets are made, for example, without limitation, by molding in a suitable tableting machine, a mixture of powdered compounds moistened with an inert liquid diluent. In an embodiment, the tablets may optionally be coated or scored, and may be formulated so as to provide slow or controlled release of the active ingredients, using, for example, without limitation, hydroxypropylmethyl cellulose in varying proportions to provide the desired release profile. In an embodiment, tablets may optionally be provided with a coating, without limitation, such as a thin film, sugar coating, or an enteric coating to provide release in parts of the gut other than the stomach. In an embodiment, processes, equipment, and toll manufacturers for tablet and capsule making are well-known in the art.

In an embodiment, capsule pharmaceutical composition can utilize either hard or soft capsules, including, without limitation, gelatin capsules or vegetarian capsules such as those made out of hydroxymethylpropylcellulose (HMPC). In an embodiment, a type of capsule is a gelatin capsule. In an embodiment, capsules may be filled using a capsule filling machine such as, without limitation, those available from commercial suppliers such as Miranda International or employing capsule manufacturing techniques well-known in the industry.

Packaging and instruments for administration may be determined by a variety of considerations, such as, without limitation, the volume of material to be administered, the conditions for storage, whether skilled healthcare practitioners will administer or patient self-compliance, the dosage regime, the geopolitical environment (e.g., exposure to extreme conditions of temperature for developing nations), and other practical considerations.

Injection devices include pen injectors, auto injectors, safety syringes, injection pumps, infusion pumps, glass prefilled syringes, plastic prefilled syringes and needle free injectors syringes may be prefilled with liquid, or may be dual chambered, for example, for use with lyophilized material. An example of a syringe for such use is the Lyo-Ject™, a dual-chamber pre-filled lyosyringe available from Vetter GmbH, Ravensburg, Germany. Another example is the Lyo Tip which is a prefilled syringe designed to conveniently deliver lyophilized formulations available from LyoTip, Inc., Camarillo, California, U.S.A. Administration by injection may be, without limitation intravenous, intramuscular, intraperitoneal, or subcutaneous, as appropriate. Administrations by non-injection route may be, without limitation, nasal, oral, ocular, dermal, or pulmonary, as appropriate.

In certain embodiments, kits can comprise, without limitation, one or more single or multi-chambered syringes (e.g., liquid syringes and lyosyringes) for administering one or more pharmaceutical composition described herein. In various embodiments, the kit can comprise pharmaceutical composition for parenteral, subcutaneous, intramuscular or IV administration, sealed in a vial under partial vacuum in a form ready for loading into a syringe and administration to a subject. In this regard, the pharmaceutical composition can be disposed therein under partial vacuum. In all of these embodiments and others, the kits can contain one or more vials in accordance with any of the foregoing, wherein each vial contains a single unit dose for administration to a subject.

The kits can comprise lyophilates, disposed as herein, that upon reconstitution provide pharmaceutical compositions in accordance therewith. In various embodiment the kits can contain a lyophilate and a sterile diluent for reconstituting the lyophilate.

The kit can comprise a combination of a pharmaceutical composition of streptogramin B and a pharmaceutical composition of streptogramin A, wherein, in an embodiment the streptogramin B is pristinamycin IA or a pharmaceutically acceptable salt thereof and the streptogramin A is flopristin or a pharmaceutically acceptable salt thereof. The kit can further comprise a package within which the combination of a pharmaceutical composition of streptogramin B and a pharmaceutical composition of streptogramin A are contained with one or more of instructions, labels, containers and other items necessary for the use of the combination of a pharmaceutical composition of streptogramin B and a pharmaceutical composition of a streptogramin A to a subject to treat an infection and/or disease when both compositions are administered together at the same time, or one composition is administered before or after the other composition.

In an embodiment, a pharmaceutical composition as described herein can be administered by any suitable route, specifically by parental (including subcutaneous, intramuscular, intravenous and intradermal) administration and/or into the interstices regions of a joint. It will also be appreciated that the preferred route will vary with the condition and age of the recipient, and the disease being treated. Methods of determining the most effective means and dosage of administration are known to those of skill in the art and will vary, without limitation, with the pharmaceutical composition used for therapy, the purpose of the therapy, and the subject being treated. Single or multiple administrations can be carried out, without limitation, the dose level and pattern being selected by the treating physician. Suitable dosage pharmaceutical compositions and methods of administering the agents are known in the art.

The pharmaceutical compositions as described herein can be used in the manufacture of medicaments and for the treatment of humans and other animals by administration in accordance with conventional procedures.

Suitable routes of administration may, for example, include oral, rectal, transmucosal, inhalation, intranasal, topical (including dermal, ophthalmic, otic), vaginal, intestinal, buccal, or sublingual administration; parenteral delivery, including intramuscular, subcutaneous, intravenous, or intramedullary injections, as well as intrathecal, epidural, direct intraventricular, intraperitoneal, intraamniotic, intranasal, or intraocular injections or injection into the joints.

The compounds can also be administered in sustained or controlled release dosage forms, including depot injections, osmotic pumps, gels, suspensions or emulsions, pills, transdermal patches, and the like, for prolonged and/or timed, pulsed administration at a predetermined rate.

The determination of effective dosage levels, that is the dosage levels necessary to achieve the desired result, can be accomplished by one skilled in the art using routine pharmacological methods. Typically, human clinical applications of products are commenced at lower dosage levels, with dosage level being increased until the desired effect is achieved. Alternatively, acceptable in vitro studies can be used to establish useful doses and routes of administration of the compositions.

Administration of the therapeutic agents in accordance with the present invention may be in a single dose, in multiple doses, in a continuous or intermittent manner, depending, for example, upon the subject's physiological condition, whether the purpose of the administration is therapeutic or prophylactic, and other factors known to skilled practitioners. The administration of the active antibacterial compounds of the invention may be essentially continuous over a preselected period of time or may be in a series of spaced doses. Both local and systemic administration is contemplated.

### Combination Therapy

By “combination therapy” it is meant that a composition described herein is administered at the same time, just prior to, or just after the administration of one or more additional therapies. The compounds provided herein can be administered alone or can be coadministered to the subject. Coadministration is meant to include simultaneous or sequential administration of the compounds individually or in combination (more than one compound). Thus, the preparations can also be combined, when desired, with other active substances (e.g. to reduce metabolic degradation or other activities). The compositions of the present invention can be delivered orally, parenterally, topically or transdermally or by inhalation, or formulated as applicator sticks, solutions, suspensions, emulsions, sachets, sprinkles, syrups, elixirs, tablets, capsules, caplets, gels, creams, ointments, foams, lotion, pastes, patches, jellies, paints, powders, solids, liquids, liposomes and aerosols. In embodiments, co-administer includes simultaneous or sequential administration of the compounds (e.g., compounds described herein) individually in addition to an additional secondary agent (e.g., additional antibiotic).

### EXAMPLES

### Example 1

### Streptogramin Combination Selection

Synergy measurement by checkerboard analysis is used to determine the impact on potency of the combination of antibiotics in comparison to their individual activities (Pillai S K, et al. “Antimicrobial combinations”. Antibiotics in Laboratory Medicine 5^{th} Ed. 2005, pages 365-440). Further, because checkerboard studies assess a wide range of combination ratios, they provide a means to comparatively assess activity for different combinations of antibiotics across different ratios.

A checkerboard analysis was used to investigate the best streptogramin B and streptogramin A combination. Novel streptogramin combinations, identified by Combination of streptogramin B with streptogramin A (CBA) ID are listed in Table 1. Known streptogramin combinations pristinamycin IA-pristinamycin IIA, quinupristin-dalfopristin, and linopristin-flopristin were included as benchmark in class comparators.

TABLE 1

| Combination ID | Streptogramin B | Streptogramin A |
|---|---|---|
| CBA1 | pristinamycin IA | flopristin |
| CBA2 | quinupristin | flopristin |
| CBA3 | quinupristin | pristinamycin IIA |
| CBA4 | pristinamycin IA | dalfopristin |
| CBA5 | linopristin | dalfopristin |
| CBA6 | linopristin | pristinamycin IIA |
| pristinamycin IA-pristinamycin IB | pristinamycin IA | pristinamycin IIA |
| quinupristin-dalfopristin | quinupristin | dalfopristin |
| linopristin-flopristin | linopristin | flopristin |

The checkerboard analysis was performed with four Neisseria gonorrhoeae strains, five Staphylococcus aureus strains, two Streptococcus pneumoniae strains, and one Haemophilus influenzae strain (see Table 2 for details of strains utilized and their antibiotic resistance profiles).

TABLE 2

| | Strain | | |
|---|---|---|---|
| Organism | ID | Isolate Name | Type |
| Neisseria | 1 | NCTC 13477 | Susceptible |
| gonorrhoeae | 2 | NCTC 13480 | Penicillin^{R}, Tetracycline^{R}, |
| | | | Ciprofloxacin^{R}, Ceftriaxone^{R} |
| | 3 | NCTC 13817 | Azithromycin^{R} |
| | 4 | NCTC 13818 | Penicillin^{R}, Tetracycline^{R}, |
| | | | Ciprofloxacin^{R}, Azithromycin^{R} |
| Staphylococcus | 5 | MMX 3873 | MSSA, Erythromycin^{R} |
| aureus | 6 | MMX 5787 | MRSA, Erythromycin^{R}, |
| | | | Clindamycin^{R}, Linezolid^{R} |
| | 7 | NRS 271 | MRSA, Linezolid^{R} |
| | 8 | MMX 3983 | MRSA, Erythromycin^{R}, |
| | | | Clindamycin^{R} |
| | 9 | MMX 3095 | MRSA, Erythromycin^{R} |
| Streptococcus | 10 | MMX 4937 | Erythromycin^{R} |
| pneumoniae | 11 | ATCC 700677 | Penicillin^{R}, Tetracycline^{R}, |
| | | | Erythromycin^{R} |
| Haemophilus | 12 | ATCC 49247 | Susceptible |
| influenzae | | | |

An R superscript following an antibiotic indicates resistance to that antibiotic;
MSSA: methicillin-susceptible S. aureus;
MRSA: methicillin-resistant S. aureus

An 8×12 checkerboard test format was used for testing each of the nine combinations. MIC assay plates were prepared using the Clinical and Laboratory Standards Institute (CLSI) broth microdilution procedures (CLSI “Performance Standards for Antimicrobial Susceptibility Testing. 28th ed.” CLSI supplement M100. CLSI, 950 West Valley Road, Suite 2500, Wayne, Pennsylvania 19087 USA, 2018; CLSI “Methods for Dilution Antimicrobial Susceptibility Tests for Bacteria That Grow Aerobically; Approved Standard-11th Edition” CLSI standard M07. CLSI, 950 West Valley Road, Suite 2500, Wayne, Pennsylvania 19087 USA, 2018; CLSI “Methods for Antimicrobial Dilution and Disk Susceptibility Testing of Infrequently Isolated or Fastidious Bacteria. 3^{rd} ed.” CLSI guideline 45. CLSI, 950 West Valley Road, Suite 2500, Wayne, Pennsylvania 19087 USA, 2016). For testing of N. gonorrhoeae, a modified medium described by the ATCC capable of supporting growth was used. This medium contains 15 g Oxoid Special Peptone, 1 g corn starch, 5 g NaCl, 4 g K₂HPO₄ and 1 g KH₂PO₄ per liter and 1% IsoVitaleX enrichment (Becton Dickinson).

Stock solutions of test articles were prepared in dimethyl sulfoxide (DMSO) at 102× the highest final concentration to be tested. Serial dilutions were made in “mother” plates, one for the horizontally diluted streptogramin component and another for the vertically diluted streptogramin component. For the horizontally diluted mother plate, the wells of standard 96-well microdilution plates (Costar) were filled with 150 μL of DMSO in Columns 2 through 12. A 300 μL aliquot of the stock solution for the test agent was added to each well in Column 1 of the plate. The automated liquid handler Biomek 2000 (Beckman Coulter, Fullerton CA) was used to make eleven 2-fold serial dilutions horizontally across each row of the plate from Columns 2-11 (left to right).

For the vertically diluted mother plate, 150 μL of DMSO was added to wells in Rows B-H (top to bottom) of a 96-well microtiter plate. Row A of this plate was filled with 300 μL each test agent at 102× the highest final concentration to be tested. Serial 2-fold dilutions were then prepared from Row B-G by hand using a multichannel pipette.

The “daughter plates” were loaded with 190 μL of test medium using the automated liquid handler Multidrop 384 (Labsystems, Helsinki, Finland). The automated liquid handler Biomek FX (Beckman Coulter, Fullerton CA) was used to transfer 2 μL of drug solution from each well of the horizontal dilution mother plate to the corresponding well in all of the daughter plates in a single step. Then a 2 μL aliquot from each well of the vertical dilution mother plate was transferred with the Biomek FX into the corresponding well of the daughter plate. Row H and Column 12 each contained serial dilutions of a single drug for determination of the MIC for the drug alone. This procedure was repeated for all test agents.

A standardized inoculum of each organism was prepared per CLSI methods. A 10 μL standardized inoculum was delivered into each well using the Biomek 2000 from low to high concentration. These inoculations yielded a final cell concentration in the daughter plates of approximately 5×10⁵ colony forming unit (CFU)/mL in each well. Thus, the wells of the daughter plates ultimately contained 190 μL of media, 2 L of one test agent, 2 μL of a second test agent, and 10 μL of inoculum.

The test format resulted in the creation of an 8×12 checkerboard where each compound was tested alone (Column 12 and Row H) and in combination at varying ratios of compound concentrations with each well of the microtiter plate containing a unique combination of concentrations of the two streptogramins. Exact concentration ranges of each compound used in the 96-well plates were based on previously obtained MIC values for each component alone against a specific strain. Ranges evaluated for each streptogramin combination and each strain are provided in the following paragraph (see Table 1 for CBA ID and Table 2 for Strain ID). For a specific combination, the first concentration range listed is for the streptogramin B component and the second concentration range listed is for the streptogramin A component. Ranges with eleven 2-fold values were diluted horizontally on the plate and ranges with seven 2-fold values were diluted vertically on the plate.

CBA1, CBA2, and linopristin-flopristin were evaluated at 0.004-4 & 0.004-0.25 μg/mL for strains 1-4 and 12 and at 0.004-4 & 0.03-2 μg/mL for strains 5-11. CBA3 was evaluated at 0.004-4 & 0.016-1 μg/mL for strains 1, 2, and 12, at 0.008-8 & 0.03-2 μg/mL for strains 3 and 4, at 0.004-4 & 0.06-4 μg/mL for strains 5, 8, and 9, and at 0.008-8 & 0.12-8 μg/mL for strains 6, 7, 10 and 11. CBA4 was evaluated at 0.004-4 & 0.008-0.5 μg/mL for strains 1, 2 and 12, at 0.016-16 & 0.06-4 μg/mL for strains 3, 4, 6, 7, 10, and 11, and at 0.004-4 & 0.03-2 μg/mL for strains 5, 8 and 9. CBA5 and CBA6 were evaluated at 0.06-4 & 0.004-4 μg/mL for strains 1-12. Pristinamycin IA-pristinamycin IIA was evaluated at 0.004-4 & 0.016-1 μg/mL for strains 1, 2, and 12, at 0.016-16 & 0.03-2 μg/mL for strains 3 and 4, at 0.004-4 & 0.06-4 μg/mL for strains 5, 8, and 9, and at 0.016-16 & 0.12-8 μg/mL for strains 6, 7, 10 and 11. Quinupristin-dalfopristin was evaluated at 0.004-4 & 0.008-0.5 μg/mL for strains 1, 2, and 12, at 0.008-8 & 0.06-4 μg/mL for strains 3, 4, 6, 7, 10 and 11, and at 0.004-4 & 0.03-2 μg/mL for strains 5, 8, and 9.

Following inoculation, plates were incubated for approximately 20 hours at 35° C. in ambient atmosphere for S. aureus and S. pneumoniae and in an atmosphere of 5% CO₂ for N. gonorrhoeae.

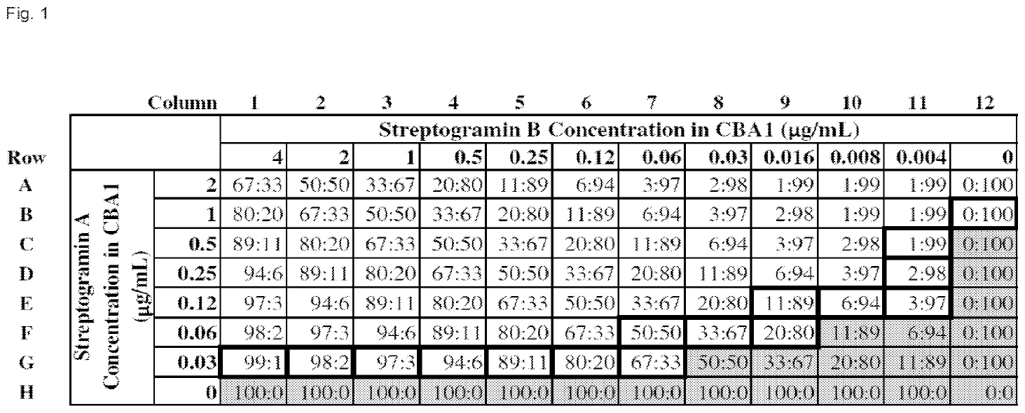

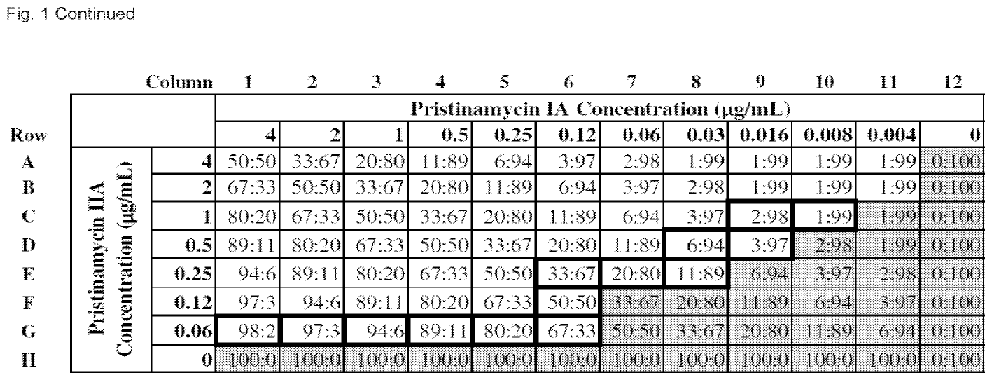

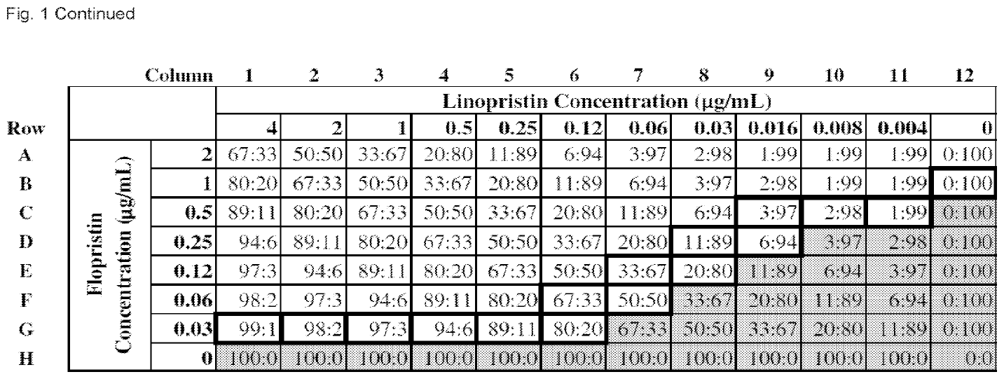

For MIC determination, plates were viewed from the bottom using a plate viewer. The MIC for a specific combination ratio was read as the lowest combined concentration of streptogramin B and streptogramin A at that ratio where visible growth of the organism was completely inhibited. An example is provided in FIG. 1, which shows the results of the checkerboard MIC testing of the streptogramin combination of CBA1 (top), pristinamycin IA with pristinamycin IIA (middle), and linopristin with flopristin (bottom) against S. aureus strain 5. The ratio value in each well indicates the concentration ratio of the streptogramin B to streptogramin A in that well. The shaded wells in the microtiter plate indicate microbial growth. The MICs of specific streptogramin concentration ratios in FIG. 1 are identified with bold borders surrounding the well. As examples, in the CBA1 panel, the MIC at a ratio of 33:67 for the streptogramin B:streptogramin A combination is located in well F8 and is 0.03:0.06 μg/mL (for a total MIC of 0.09 μg/mL) and the MIC at a ratio of 20:80 for the streptogramin B:streptogramin A combination is located in well F9 and is 0.016:0.06 μg/mL (for a total MIC of 0.076 μg/mL).

The checkerboard MIC results were used to compare the nine streptogramin combinations listed in Table 1 across various ratios, species, and strains. A comparative analysis of MICs obtained at the 33:67 and 67:33 ratios is provided below. Across the nine combinations evaluated, in every species and in every strain tested, the novel CBA1 combination consistently effected the lowest MIC values demonstrating an unexpected finding of improved potency over all other streptogramin combinations.

Table 3 shows the MIC values of the nine combinations of streptogramins each tested at a streptogramin B:streptogramin A ratio of 33:67 in Neisseria gonorrhoeae, Staphylococcus aureus, Streptococcus pneumoniae, and Haemophilus influenzae. The combination with the lowest mean MIC was CBA1 with a mean MIC value of 0.19 μg/mL; while PIA-PIIA, quinupristin-dalfopristin, and linopristin-flopristin had mean MIC values of 0.43 μg/mL, 0.81 μg/mL, and 0.31 μg/mL, respectively.

TABLE 3
MIC (μg/mL) for the Nine Streptogramin Combinations at a Streptogramin B:Streptogramin A
Ratio of 33:67

| | | Streptogramin Combination | | | | | | | | |
|---|---|---|---|---|---|---|---|---|---|---|
| | St. | CBA | CBA | CBA | CBA | CBA | CBA | PIA- | | |
| Species | ID | 1 | 2 | 3 | 4 | 5 | 6 | PIIA | Q-D | L-F |
| N. gonorrhoeae | 1 | 0.024 | 0.024 | 0.18 | 0.09 | ≤0.18 | ≤0.18 | 0.09 | 0.18 | 0.024 |
| | 2 | 0.09 | 0.18 | 1.5 | 0.37 | 0.75 | 0.37 | 0.37 | 0.75 | 0.09 |
| | 3 | 0.09 | 0.18 | 0.18 | 0.37 | 0.75 | 0.37 | 0.37 | 0.37 | 0.09 |
| | 4 | 0.18 | 0.18 | 0.37 | 0.37 | 0.75 | 0.75 | 0.18 | 0.75 | 0.18 |
| S. aureus | 5 | 0.09 | 0.75 | 1.5 | 0.37 | 0.37 | 0.37 | 0.37 | 1.5 | 0.18 |
| | 6 | 0.75 | 1.5 | 0.75 | 3 | 3 | 3 | 1.5 | 1.5 | 1.5 |
| | 7 | 0.18 | 0.75 | 0.37 | 0.37 | 0.75 | 0.37 | 0.18 | 0.37 | 0.18 |
| | 8 | 0.37 | 0.75 | 1.5 | 1.5 | 1.5 | 1.5 | 0.75 | 1.5 | 0.37 |
| | 9 | 0.09 | 0.37 | 0.75 | 0.18 | 0.37 | 0.37 | 0.09 | 0.75 | 0.18 |
| S. pneumoniae | 10 | 0.09 | 0.75 | 0.37 | 0.18 | 0.37 | 0.37 | ≤0.18 | 0.18 | 0.18 |
| | 11 | 0.18 | 1.5 | 0.37 | 0.37 | 0.75 | 0.75 | 0.37 | 0.37 | 0.37 |
| H. influenzae | 12 | 0.18 | 0.18 | 1.5 | 0.75 | 1.5 | 1.5 | 0.75 | >0.75 | 0.37 |

| | | | | | | | | | |
|---|---|---|---|---|---|---|---|---|---|
| Mean MIC | 0.19 | 0.59 | 0.78 | 0.66 | 0.92 | 0.83 | 0.43 | 0.81 | 0.31 |
| Rank | 1 | 4 | 6 | 5 | 9 | 8 | 3 | 7 | 2 |

≤indicates one component at limit of detection based on ranges tested, and therefore MIC is ≤ value shown. For calculation of mean, the value shown was used.
>indicates MIC is greater than the range of tested values, and therefore > value is shown. For calculation of mean, the MIC was set at the next 2-fold higher value.
St. ID = strain identification in Table 2, CBA code in Table 1, PIA-PIIA = pristinamycin IA-pristinamycin IIA, Q-D = quinupristin-dalfopristin, L-F = linopristin-flopristin.

Table 4 shows ranking of the MIC values of the nine combinations of streptogramins each tested at a streptogramin B:streptogramin A ratio of 33:67 in each strain of Neisseria gonorrhoeae, Staphylococcus aureus, Streptococcus pneumoniae, and Haemophilus influenzae. For each strain tested and across all strains, CBA1 consistently had the best ranking (Rank 1).

Table 5 shows the MIC values of the nine combinations of streptogramins each tested at a streptogramin B:streptogramin A ratio of 67:33 in Neisseria gonorrhoeae, Staphylococcus aureus, Streptococcus pneumoniae, and Haemophilus influenzae. The combination with the lowest mean MIC was CBA1 with a mean MIC value of 0.23 μg/mL; while PIA-PIIA, quinupristin-dalfopristin, and linopristin-flopristin had mean MIC values of 0.54 μg/mL, 0.81 μg/mL, and 0.42 μg/mL, respectively.

Table 6 shows ranking of the MIC values of the nine combinations of streptogramins each tested at a streptogramin B:streptogramin A ratio of 67:33 in each strain of Neisseria gonorrhoeae, Staphylococcus aureus, Streptococcus pneumoniae, and Haemophilus influenzae. For each strain tested and across all strains, CBA1 consistently had the best ranking (Rank 1).

TABLE 4
Ranking of MIC (μg/mL) Values by Strain for the Nine Streptogramin Combinations at a
Streptogramin B:Streptogramin A Ratio of 33:67

| | St. | CBA | CBA | CBA | CBA | CBA | CBA | PIA- | | |
|---|---|---|---|---|---|---|---|---|---|---|
| Species | ID | 1 | 2 | 3 | 4 | 5 | 6 | PIIA | Q-D | L-F |
| N. gonorrhoeae | 1 | 1 | 1 | 6 | 4 | 6 | 6 | 4 | 6 | 1 |
| | 2 | 1 | 3 | 9 | 4 | 7 | 4 | 4 | 7 | 1 |
| | 3 | 1 | 3 | 3 | 5 | 9 | 5 | 5 | 5 | 1 |
| | 4 | 1 | 1 | 5 | 5 | 7 | 7 | 1 | 7 | 1 |
| S. aureus | 5 | 1 | 7 | 8 | 3 | 3 | 3 | 3 | 8 | 2 |
| | 6 | 1 | 3 | 1 | 7 | 7 | 7 | 3 | 3 | 3 |
| | 7 | 1 | 8 | 4 | 4 | 8 | 4 | 1 | 4 | 1 |
| | 8 | 1 | 3 | 5 | 5 | 5 | 5 | 3 | 5 | 1 |
| | 9 | 1 | 5 | 8 | 3 | 5 | 5 | 1 | 8 | 3 |
| S. pneumoniae | 10 | 1 | 9 | 6 | 2 | 6 | 6 | 2 | 2 | 2 |
| | 11 | 1 | 9 | 2 | 2 | 7 | 7 | 2 | 2 | 2 |
| H. influenzae | 12 | 1 | 1 | 6 | 4 | 6 | 6 | 4 | 6 | 3 |

| | | | | | | | | | |
|---|---|---|---|---|---|---|---|---|---|
| Sum | 12 | 53 | 63 | 48 | 76 | 65 | 33 | 63 | 21 |
| Total Ranking | 1 | 5 | 6 | 4 | 9 | 8 | 3 | 6 | 2 |

Shaded cell indicates one component at limit of detection based on ranges tested.
St. ID = strain identification in Table 2, CBA code in Table 1, PIA-PIIA = pristinamycin IA-pristinamycin IIA, Q-D = quinupristin-dalfopristin, L-F = linopristin-flopristin.

TABLE 5
MIC (μg/mL) for the Nine Streptogramin Combinations at a Streptogramin B:Streptogramin A
Ratio of 67:33

| | St. | CBA | CBA | CBA | CBA | CBA | CBA | PIA- | | |
|---|---|---|---|---|---|---|---|---|---|---|
| Species | ID | 1 | 2 | 3 | 4 | 5 | 6 | PIIA | Q-D | L-F |
| N. gonorrhoeae | 1 | 0.046 | 0.046 | 0.18 | 0.18 | 0.18 | 0.18 | 0.18 | 0.18 | 0.046 |
| | 2 | 0.18 | 0.37 | 0.75 | 0.75 | 0.75 | 0.75 | 0.37 | 0.75 | 0.18 |
| | 3 | 0.09 | 0.18 | 0.18 | 0.75 | 0.75 | 0.75 | 0.37 | 0.37 | 0.18 |
| | 4 | 0.18 | 0.37 | 0.75 | 0.75 | 1.5 | 0.75 | 0.37 | 0.75 | 0.37 |
| S. aureus | 5 | 0.09 | 0.75 | 1.5 | 0.37 | 0.75 | 0.37 | 0.18 | 1.5 | 0.18 |
| | 6 | 0.75 | 1.5 | 1.5 | 3 | 3 | 3 | 1.5 | 1.5 | 1.5 |
| | 7 | 0.09 | 0.75 | ≤0.37 | 0.37 | 0.75 | 0.37 | ≤0.37 | 0.37 | 0.37 |
| | 8 | 0.75 | 0.75 | 1.5 | 1.5 | 3 | 1.5 | 1.5 | 1.5 | 0.75 |
| | 9 | ≤0.09 | 0.37 | 0.75 | 0.37 | 0.37 | 0.37 | 0.18 | 0.75 | 0.18 |
| S. pneumoniae | 10 | ≤0.09 | 0.75 | ≤0.37 | 0.18 | 0.37 | 0.18 | ≤0.37 | 0.18 | 0.18 |
| | 11 | 0.09 | 1.5 | ≤0.37 | 0.37 | 0.75 | 0.75 | ≤0.37 | 0.37 | 0.37 |
| H. influenzae | 12 | 0.37 | 0.37 | 1.5 | 1.5 | 3 | 1.5 | 0.75 | 1.5 | 0.75 |

| | | | | | | | | | |
|---|---|---|---|---|---|---|---|---|---|
| Mean MIC | 0.23 | 0.64 | 0.81 | 0.84 | 1.26 | 0.87 | 0.54 | 0.81 | 0.42 |
| Rank | 1 | 4 | 5 | 7 | 9 | 8 | 3 | 5 | 2 |

≤indicates one component at limit of detection based on ranges tested, and therefore MIC is ≤ value shown. For calculation of mean, the value shown was used.
St. ID = strain identification in Table 2, CBA code in Table 1, PIA-PIIA = pristinamycin IA-pristinamycin IIA, Q-D = quinupristin-dalfopristin, L-F = flopristin.

TABLE 6
Ranking of MIC (μg/mL) Values by Strain for the Nine Streptogramin Combinations at a
Streptogramin B:Streptogramin A Ratio of 67:33

| | St. | CBA | CBA | CBA | CBA | CBA | CBA | PIA- | | |
|---|---|---|---|---|---|---|---|---|---|---|
| Species | ID | 1 | 2 | 3 | 4 | 5 | 6 | PIIA | Q-D | L-F |
| N. gonorrhoeae | 1 | 1 | 1 | 4 | 4 | 4 | 4 | 4 | 4 | 1 |
| | 2 | 1 | 3 | 5 | 5 | 5 | 5 | 3 | 5 | 1 |
| | 3 | 1 | 2 | 2 | 7 | 7 | 7 | 5 | 5 | 2 |
| | 4 | 1 | 2 | 5 | 5 | 9 | 5 | 2 | 5 | 2 |
| S. aureus | 5 | 1 | 6 | 8 | 4 | 6 | 4 | 2 | 8 | 2 |
| | 6 | 1 | 2 | 2 | 7 | 7 | 7 | 2 | 2 | 2 |
| | 7 | 1 | 8 | 2 | 2 | 8 | 2 | 2 | 2 | 2 |
| | 8 | 1 | 1 | 4 | 4 | 8 | 8 | 4 | 4 | 1 |
| | 9 | 1 | 4 | 8 | 4 | 4 | 4 | 2 | 8 | 2 |
| S. pneumoniae | 10 | 1 | 9 | 6 | 2 | 6 | 2 | 6 | 2 | 2 |
| | 11 | 1 | 9 | 2 | 2 | 7 | 7 | 2 | 2 | 2 |
| H. influenzae | 12 | 1 | 1 | 5 | 5 | 9 | 5 | 3 | 5 | 3 |

| | | | | | | | | | |
|---|---|---|---|---|---|---|---|---|---|
| Sum | 12 | 48 | 53 | 51 | 80 | 60 | 37 | 52 | 22 |
| Total Ranking | 1 | 4 | 7 | 5 | 9 | 8 | 3 | 6 | 2 |

Shaded cell indicates one component at limit of detection based on ranges tested.
St. ID = strain identification in Table 2, CBA code in Table 1, PIA-PIIA = pristinamycin IA-pristinamycin IIA, Q-D = quinupristin-dalfopristin, L-F = flopristin.

Similar results in ranking were observed at other streptogramin B:streptogramin A ratios between 0:100 and 99:1. As an illustration, the MIC values at each ratio for CBA1, pristinamycin IA-pristinamycin IIA, and linopristin-flopristin that were depicted in FIG. 1 against S. aureus strain 5 are presented in Table 7. In the majority of ratios evaluated with activity above the limit of detection, the novel CBA1 combination was 2 to 4-fold more potent than the known streptogramins combinations: pristinamycin IA-pristinamycin IIA and linopristin-flopristin which only differ from CBA1 in one component. In no instance, was the MIC of either pristinamycin IA-pristinamycin IIA or linopristin-flopristin lower than that of CBA1. Collectively, these example data illustrate an unexpected result of improved potency for this novel combination CBA1 (pristinamycin IA and flopristin) consistently over a wide range of ratios as compared to known combinations containing pristinamycin IA or flopristin and other new streptogramin combinations.

TABLE 7
Checkerboard MICs of CBA1, Pristinamycin IA-Pristinamycin IIA,
and Linopristin-Flopristin for Streptogramin B to Streptogramin
A Ratios of 0:100 to 99:1 against S. aureus strain 5

| | | | | Fold Increase in | |
|---|---|---|---|---|---|
| B:A | MIC (μg/mL) | | | Potency of CBA1 Over: | |
| Ratio | CBA1 | PIA-PIIA | L-F | PIA-PIIA | L-F |
| 0:100 | 1 | >4 | 1 | >4 | 1 |
| 1:99 | 0.504 | 1.008 | 0.504 | 2 | 1 |
| 2:98 | 0.29 | 1.016 | 0.508 | 4 | 2 |
| 3:97 | 0.124 | 0.516 | 0.516 | 4 | 4 |
| 6:94 | 0.128 | 0.53 | 0.266 | 4 | 2 |
| 11:89 | 0.136 | 0.28 | 0.28 | 2 | 2 |
| 20:80 | 0.076 | 0.31 | 0.15 | 4 | 2 |
| 33:67 | 0.09 | 0.37 | 0.18 | 4 | 2 |
| 50:50 | 0.12 | 0.24 | 0.12 | 2 | 1 |
| 67:33 | 0.09 | 0.18 | 0.18 | 2 | 2 |
| 80:20 | ≤0.15 | ≤0.31 | 0.15 | NC | NC |
| 89:11 | ≤0.28 | ≤0.56 | ≤0.28 | NC | NC |
| 94:6 | ≤0.53 | ≤1.06 | ≤0.53 | NC | NC |
| 97:3 | ≤1.03 | ≤2.06 | ≤1.03 | NC | NC |
| 98:2 | ≤2.03 | ≤4.06 | ≤2.03 | NC | NC |
| 99:1 | ≤4.03 | ND | ≤4.03 | NC | NC |

Ratio B:A is the ratio of the concentration of streptogramin B to the concentration of streptogramin A; PIA-PIIA: pristinamycin IA-pristinamycin IIA; L-F: linopristin-flopristin. Fold increase in potency is calculated by dividing MIC of PIA-PIIA or L-F by MIC of CBA1. NC: not calculated due to MIC values at limit of detection; ND: not determined.

### Example 2

### Antibacterial Profile of CBA1 Across Streptogramin Component Ratios

The effect of varying the streptogramin B:streptogramin A ratios in CBA1 by weight on minimum inhibitory concentrations (MIC) and minimum bactericidal concentrations (MBC) was investigated in a panel of Streptococcus pneumoniae, Neisseria gonorrhoeae, and Staphylococcus aureus enriched for antibiotic resistant strains. The streptogramin B:streptogramin A ratios in CBA1 investigated were 100:0, 90:10, 70:30, 50:50, 30:70, 10:90, and 0:100 by weight. Pristinamycin IA-pristinamycin IIA was included as an in-class comparator at the same ratios. A panel of Mycoplasma genitalium clinical isolates and strains were similarly evaluated with CBA1 at ratios of 100:0, 70:30, 30:70, and 0:100.

MICs were determined for each streptogramin B:streptogramin A ratio using the CLSI broth microdilution procedure (CLSI “Performance Standards for Antimicrobial Susceptibility Testing. 28th ed.” CLSI supplement M100. CLSI, 950 West Valley Road, Suite 2500, Wayne, Pennsylvania 19087 USA, 2018; CLSI “Methods for Dilution Antimicrobial Susceptibility Tests for Bacteria That Grow Aerobically; Approved Standard—11th Edition” CLSI standard M07. CLSI, 950 West Valley Road, Suite 2500, Wayne, Pennsylvania 19087 USA, 2018. For testing of N. gonorrhoeae, a modified medium was utilized as described in Example 1 above. For testing of M. genitalium, mycoplasma, CLSI methods for the susceptibility testing of mycoplasmas were followed (CLSI “Methods for antimicrobial susceptibility testing of human mycoplasmas” Approved Guideline M43-A. CLSI, 950 West Valley Road, Suite 2500, Wayne, Pennsylvania 19087 USA, 2011).

Stock solutions for the streptogramins were prepared at the designated streptogramin B:streptogramin A ratios in DMSO at 101× the final concentration. Eleven serial two-fold dilutions of test articles were made in DMSO and added to wells of a standard 96-well microdilution plate (Costar 3795) containing medium appropriate for the species being tested. Each row of the microtiter plate contained the two-fold dilution series in Columns 1 through 11. The wells of Column 12 contained no drug and served as the organism growth control wells. A standardized inoculum of each organism was prepared per CLSI methods targeting a final inoculum concentration of approximately 5×10⁵ CFU/mL. The final volume in the wells during susceptibility testing was 202 μL. After incubation for approximately 20 hours at 35° C. in ambient atmosphere for S. aureus and S. pneumoniae and in an atmosphere of 5% CO₂ for N. gonorrhoeae, plates were viewed from the bottom using a plate viewer. MIC values were read as the lowest concentration of test article where visible growth of the organism was completely inhibited.

The majority of strains selected for inclusion in the testing panels were antibiotic resistant. Among the 5 S. pneumoniae strains, 4 were resistant to macrolides and one of these was also resistant to penicillin and tetracycline. The panel of N. gonorrhoeae strains selected were predominantly multi-drug resistant or extensively-drug resistant strains from the WHO and CDC Antimicrobial Resistance Isolate Bank collections. These included isolates with resistance to penicillin, fluoroquinolones, tetracyclines, macrolides, and extended-spectrum cephalosporins. Among the 15 S. aureus strains, 11 were resistant to methicillin, 10 were resistant to macrolides, and 2 were resistant to linezolid. Breakpoints have not been established for M. genitalium. However, among the 10 M. genitalium strains and isolates tested, 3 had elevated MICs for azithromycin and moxifloxacin.

Table 8 shows the CBA1 MIC values for the various ratios of streptogramin B:streptogramin A in a panel of multi-drug resistant and extensively-drug resistant Neisseria gonorrhoeae. Azithromycin was used as a comparator to evaluate activity against macrolide-resistant N. gonorrhoeae. Across streptogramin B:streptogramin A ratios from 90:10 to 0:100, CBA1 had significant antibacterial activity against N. gonorrhoeae. In all cases, little to no antibacterial activity was observed with the streptogramin B component alone (i.e., CBA1 at a 100:0 ratio). However, antibacterial activity was observed with the streptogramin A component alone (i.e., CBA1 at a 0:100). CBA1 maintained potent activity against macrolide-resistant strains and there was no evidence of cross-resistance between CBA1 and macrolides.

TABLE 8
CBA1 MICs against Neisseria gonorrhoeae Across a Range of Streptogramin B:Streptogramin A Ratios

| | MIC (μg/mL) | | | | | | | |
|---|---|---|---|---|---|---|---|---|
| | CBA1 | CBA1 | CBA1 | CBA1 | CBA1 | CBA1 | CBA1 | |
| Strain | 100:0 | 90:10 | 70:30 | 50:50 | 30:70 | 10:90 | 0:100 | AZI* |
| ATCC 49226 | >64 | 0.123 | 0.5 | 0.24 | 0.128 | 0.123 | 0.12 | 0.5 |
| WHO F | 32 | 0.123 | 0.03 | 0.03 | 0.016 | 0.015 | 0.015 | 0.12 |
| WHO G | >64 | 0.255 | 0.128 | 0.12 | 0.058 | 0.06 | 0.12 | 0.25 |
| WHO K | >64 | 0.5 | 0.255 | 0.12 | 0.128 | 0.123 | 0.25 | 0.5 |
| WHO L | 64 | 0.5 | 0.128 | 0.12 | 0.128 | 0.123 | 0.12 | 0.5 |
| WHO M | 64 | 0.5 | 0.255 | 0.12 | 0.128 | 0.123 | 0.12 | 0.5 |
| WHO N | >64 | 0.255 | 0.128 | 0.06 | 0.058 | 0.06 | 0.12 | 0.25 |
| WHO O | 64 | 0.5 | 0.255 | 0.12 | 0.128 | 0.123 | 0.12 | 0.5 |
| WHO W | 64 | 0.5 | 0.128 | 0.12 | 0.128 | 0.123 | 0.12 | 0.5 |
| WHO X | >64 | 0.5 | 0.255 | 0.12 | 0.128 | 0.123 | 0.12 | 1 |
| WHO Y | 32 | 1 | 0.255 | 0.24 | 0.255 | 0.255 | 0.12 | 1 |
| CDC 0194 | 32 | 0.255 | 0.128 | 0.06 | 0.058 | 0.06 | 0.06 | 0.5 |
| WHO P | >64 | 2 | 1 | 0.5 | 0.5 | 0.5 | 0.5 | 8 |
| WHO U | >64 | 0.5 | 0.128 | 0.12 | 0.128 | 0.123 | 0.12 | 8 |
| WHO V | >64 | 0.5 | 0.255 | 0.24 | 0.128 | 0.255 | 0.25 | >16 |
| MMX 6921 | >64 | 0.5 | 0.255 | 0.12 | 0.128 | 0.123 | 0.12 | 16 |
| CDC 0167 | >64 | 0.123 | 0.058 | 0.03 | 0.03 | 0.03 | 0.03 | 16 |
| CDC 0175 | >64 | 0.123 | 0.058 | 0.06 | 0.03 | 0.03 | 0.03 | 16 |
| CDC 0177 | >64 | 0.255 | 0.058 | 0.06 | 0.058 | 0.06 | 0.06 | 4 |
| CDC 0181 | >64 | 0.255 | 0.128 | 0.12 | 0.058 | 0.123 | 0.12 | >16 |

*Strains with MICs >1 are considered non-susceptible to macrolides

Table 9 shows the fold of increase in potency of CBA1 against multiple strains of N. gonorrhoeae compared to pristinamycin IA-pristinamycin IIA at each of the same streptogramin B:streptogramin A ratios as determined by dividing the MIC of pristinamycin IA-pristinamycin IIA by the MIC of CBA1. CBA1 was mostly 4- to 8-fold more potent than pristinamycin IA-pristinamycin IIA across all ratios and all strains evaluated.

TABLE 9
Fold of Increase in Potency of CBA1 over Pristinamycin
IA-Pristinamycin IIA against Neisseria gonorrhoeae Across
a Range of Streptogramin B:Streptogramin A Ratios

| | MIC (μg/mL) | | | | |
|---|---|---|---|---|---|
| Strain | 90:10 | 70:30 | 50:50 | 30:70 | 10:90 |
| ATCC 49226 | 33 | 2 | 4 | 4 | 4 |
| WHO F | 4 | 9 | 4 | 8 | 8 |
| WHO G | 8 | 4 | 4 | 9 | 8 |
| WHO K | 8 | 8 | 8 | 8 | 8 |
| WHO L | 8 | 8 | 4 | 4 | 4 |
| WHO M | 8 | 4 | 4 | 4 | 4 |
| WHO N | 8 | 8 | 8 | 9 | 8 |
| WHO O | 8 | 4 | 4 | 4 | 4 |
| WHO W | 4 | 8 | 4 | 4 | 4 |
| WHO X | 8 | 8 | 8 | 8 | 8 |
| WHO Y | 4 | 8 | 4 | 4 | 4 |
| CDC 0194 | 4 | 4 | 4 | 4 | 4 |
| WHO P | 4 | 4 | 4 | 4 | 4 |
| WHO U | 4 | 8 | 4 | 4 | 4 |
| WHO V | 8 | 8 | 4 | 8 | 4 |
| MMX 6921 | 16 | 8 | 8 | 8 | 8 |
| CDC 0167 | 8 | 9 | 8 | 9 | 9 |
| CDC 0175 | 8 | 9 | 4 | 9 | 9 |
| CDC 0177 | 8 | 9 | 4 | 4 | 4 |
| CDC 0181 | 8 | 8 | 4 | 9 | 4 |

Fold of increase in potency calculated by dividing MIC of pristinamycin IA-pristinamycin IIA by MIC of CBA1.

In MBC assays, CBA1 was bactericidal for N. gonorrhoeae, as determined by an MBC divided by MIC value of ≤4. At streptogramin B:streptogramin A ratios in CBA1 from 90:10 to 0:100, CBA1 was bactericidal against all (20 out of the 20) N. gonorrhoeae strains. Pristinamycin IA-pristinamycin IIA demonstrated bactericidal activity against N. gonorrhoeae but only at higher concentrations, due to its decreased potency. The MBC required to inhibit 90% of all strains tested (MBC₉₀) for CBA1 ranged from 0.25 to 1 μg/mL across ratios from 90:10 to 10:90. In contrast, the MBC₉₀ for pristinamycin IA-pristinamycin IIA against N. gonorrhoeae ranged from 1 to 4 μg/mL across the same ratios. These differences in potency between the two combinations can be clinically meaningful if systemic exposure to reach above MIC or MBC of a combination cannot be achieved following certain routes of administration.

Table 10 shows the MIC values for CBA1 at various ratios of streptogramin B:streptogramin A in Streptococcus pneumoniae strains. Across the various ratios of CBA1, this novel combination demonstrated excellent antibacterial activity against S. pneumoniae. In all cases, the MIC values were much higher for incubations with only one component alone, i.e., CBA1 at ratios of 100:0 or 0:100, however, some antibacterial activity was still observed. These data demonstrate the synergism of the streptogramin combination in CBA1.

TABLE 10
CBA1 MICs against Streptococcus pneumoniae Across a
Range of Streptogramin B:Streptogramin A Ratios

| | MIC (μg/mL) | | | | | | |
|---|---|---|---|---|---|---|---|
| | CBA1 | CBA1 | CBA1 | CBA1 | CBA1 | CBA1 | CBA1 |
| Strain | 100:0 | 90:10 | 70:30 | 50:50 | 30:70 | 10:90 | 0:100 |
| ATCC | 1 | 0.25 | 0.125 | 0.06 | 0.125 | 0.125 | 2 |
| 49619 | | | | | | | |
| MMX 3031 | 0.5 | 0.125 | 0.06 | 0.03 | 0.06 | 0.06 | 4 |
| MMX 3033 | 2 | 0.25 | 0.125 | 0.125 | 0.125 | 0.25 | 4 |
| ATCC | 32 | 0.5 | 0.25 | 0.25 | 0.25 | 0.25 | 8 |
| 700677 | | | | | | | |
| MMX 4937 | 1 | 0.25 | 0.125 | 0.125 | 0.125 | 0.125 | 2 |

Table 11 shows the fold of increase in potency of CBA1 against S. pneumoniae compared to pristinamycin IA-pristinamycin IIA at each of the same streptogramin B:streptogramin A ratios as determined by dividing the MIC of pristinamycin IA-pristinamycin IIA by the MIC of that of CBA1. Overall, CBA1 was more potent than pristinamycin IA-pristinamycin IIA across all ratios evaluated, particularly against macrolide-resistant strains MMX 3031, MMX 3033, and ATCC 700677.

TABLE 11
Fold of Increase in Potency of CBA1 over Pristinamycin IA-
Pristinamycin IIA against Streptococcus pneumoniae Across
a Range of Streptogramin B:Streptogramin A Ratios

| | MIC (μg/mL) | | | | |
|---|---|---|---|---|---|
| Strain | 90:10 | 70:30 | 50:50 | 30:70 | 10:90 |
| ATCC 49619 | 0.5 | ≤0.5 | ≤1 | ≤0.5 | 1 |
| MMX 3031 | 8 | 8 | 17 | 4 | 8 |
| MMX 3033 | 2 | 2 | 2 | 4 | 2 |
| ATCC 700677 | 2 | 2 | 2 | 2 | 4 |
| MMX 4937 | 1 | 1 | 1 | 1 | 2 |

Fold of increase in potency calculated by dividing pristinamycin IA-pristinamycin IIA MIC by CBA1 MIC.

In MBC assays, CBA1 was bactericidal against Streptococcus pneumoniae across all streptogramin B:streptogramin A ratios as determined by an MBC divided by MIC value of ≤4. Across streptogramin B:streptogramin A ratios of 90:10, 70:30, 50:50, 30:70, 10:90, and 0:100, CBA1 was bactericidal against 5 out of the 5 S. pneumoniae strains. With only the streptogramin B component (i.e., a ratio of 100:0 in CBA1), it was cidal against only 2 out of the 4 S. pneumoniae strains evaluated.

Table 12 shows the CBA1 MIC values at various ratios of streptogramin B:streptogramin A against Staphylococcus aureus strains. Across the ratios from 90:10 to 0:100, CBA1 had significant antibacterial activity against S. aureus. The activity was greater at ratios where both streptogramin A and streptogramin B components were present, indicating synergism. In all cases, higher MIC values were obtained with the streptogramin B component alone (i.e., CBA1 ratio of 100:0) including no measurable antibacterial activity in certain strains. On the other hand, better antibacterial activity was observed with the streptogramin A component alone (i.e., CBA1 at a 0:100 ratio).

TABLE 12
CBA1 MICs against Staphylococcus aureus Across a Range of Streptogramin B:Streptogramin A Ratios

| | MIC (μg/mL) | | | | | | |
|---|---|---|---|---|---|---|---|
| | CBA1 | CBA1 | CBA1 | CBA1 | CBA1 | CBA1 | CBA1 |
| Strain | 100:0 | 90:10 | 70:30 | 50:50 | 30:70 | 10:90 | 0:100 |
| ATCC 29213 | 8 | 0.255 | 0.128 | 0.12 | 0.128 | 0.123 | 1 |
| MMX 3885 | 8 | 0.255 | 0.128 | 0.06 | 0.058 | 0.123 | 0.5 |
| MMX 3098 | 8 | 0.255 | 0.128 | 0.06 | 0.058 | 0.123 | 1 |
| MMX 2336 | 8 | 0.255 | 0.128 | 0.06 | 0.058 | 0.123 | 0.5 |
| NRS 271 | 8 | 0.5 | 0.255 | 0.12 | 0.128 | 0.123 | 2 |
| ATCC BAA-976 | 8 | 0.255 | 0.128 | 0.12 | 0.128 | 0.123 | 0.5 |
| ATCC BAA 977 | 16 | 0.5 | 0.255 | 0.12 | 0.128 | 0.123 | 1 |
| MMX 3035 | >64 | 0.5 | 0.255 | 0.24 | 0.255 | 0.255 | 0.5 |
| MMX 4670 | >64 | 0.255 | 0.128 | 0.12 | 0.128 | 0.123 | 0.25 |
| MMX 3037 | >64 | 0.123 | 0.058 | 0.03 | 0.03 | 0.06 | 0.25 |
| MMX 3983 | >64 | 0.5 | 0.255 | 0.24 | 0.255 | 0.255 | 0.5 |
| MMX 5787 | >64 | 1 | 1 | 1 | 0.5 | 1 | 2 |
| NRS 382 | >64 | 1 | 0.5 | 0.5 | 0.5 | 0.5 | 1 |
| NRS 384 | 8 | 0.255 | 0.128 | 0.12 | 0.128 | 0.123 | 1 |
| MMX 3095 | 8 | 0.255 | 0.128 | 0.12 | 0.128 | 0.123 | 1 |

Table 13 shows the fold of increase in potency of CBA1 against S. aureus compared to pristinamycin IA-pristinamycin IIA at each of the same streptogramin B:streptogramin A ratios as determined by dividing the MIC of pristinamycin IA-pristinamycin IIA by the MIC of CBA1. Against the majority of strains, CBA1 was more potent than pristinamycin IA-pristinamycin IIA, particularly against the strains that were both methicillin- and macrolide-resistant (the 8 strains on the bottom half of the table). The MIC that inhibits 90% of isolates tested (MIC₉₀) ranged from 0.12 to 0.5 μg/mL across the ratios from 90:10 to 10:90 for CBA1, whereas the MIC₉₀ of pristinamycin IA-pristinamycin IIA against S. aureus ranged from 2 to 8 g/mL across ratios from 90:10 to 10:90, which represents a 16-fold increased potency for CBA1 over pristinamycin IA-pristinamycin IIA based on MIC₉₀ values.

TABLE 13
Fold of Increase in Potency of CBA1 over Pristinamycin
IA-Pristinamycin IIA against Staphylococcus aureus
Across a Range of Streptogramin B:Streptogramin
A Ratios MIC (μg/mL)

| | MIC (μg/mL) | | | | |
|---|---|---|---|---|---|
| Strain | 90:10 | 70:30 | 50:50 | 30:70 | 10:90 |
| ATCC 29213 | 4 | 2 | 2 | 2 | 2 |
| MMX 3885 | 2 | 1 | 2 | 2 | 1 |
| MMX 3098 | 1 | 1 | 2 | 2 | 1 |
| MMX 2336 | 1 | 1 | 2 | 2 | 1 |
| NRS 271 | 1 | 1 | 2 | 2 | 2 |
| ATCC BAA-976 | 1 | 1 | 1 | 1 | 1 |
| ATCC BAA 977 | 1 | 1 | 2 | 2 | 2 |
| MMX 3035 | 8 | 8 | 8 | 8 | 8 |
| MMX 4670 | 2 | 4 | 4 | 4 | 4 |
| MMX 3037 | 2 | ≤1 | ≤1 | ≤0.5 | 2 |
| MMX 3983 | 4 | 4 | 4 | 4 | 4 |
| MMX 5787 | 8 | 4 | 4 | 8 | 4 |
| NRS 382 | 8 | 4 | 4 | 4 | 4 |
| NRS 384 | 2 | 1 | 1 | 1 | 2 |
| MMX 3095 | 2 | 2 | 1 | 1 | 2 |

Fold of increase in potency calculated by dividing MIC of pristinamycin IA-pristinamycin IIA by MIC of CBA1.

In MBC assays, CBA1 at streptogramin B:streptogramin A ratios from 90:10 to 10:90 demonstrated bactericidal activity against the majority of S. aureus strains (60% to 73% across ratios) based on comparison of MBC values to MIC values as shown in Table 14. Bactericidal activity of CBA1 was similar regardless of the ratio. In contrast, pristinamycin IA-pristinamycin IIA combinations exhibited primarily bacteriostatic activity against S. aureus with MBC divided by MIC values ≥8 against the majority of strains. At the clinically approved dose ratio for pristinamycin IA-pristinamycin IIA of 30:70, there was no evidence of bactericidal activity (0%) for this streptogramin combination in S. aureus.

TABLE 14
Bactericidal Activity of CBA1 and Pristinamycin IA-Pristinamycin
IIA (PIA-PIIA) against Staphylococcus aureus Across
a Range of Streptogramin B:Streptogramin A Ratios
CBA1 Bactericidal Activity against S. aureus
n/N

| CBA1 | CBA1 | CBA1 | CBA1 | CBA1 |
|---|---|---|---|---|
| 90:10 | 70:30 | 50:50 | 30:70 | 10:90 |
| 10/15 | 9/15 | 11/15 | 10/15 | 11/15 |
| (67%) | (60%) | (73%) | (67%) | (73%) |

PIA-PIIA Bactericidal Activity against S. aureus
n/N

| PIA-PIIA | PIA-PIIA | PIA-PIIA | PIA-PIIA | PIA-PIIA |
|---|---|---|---|---|
| 90:10 | 70:30 | 50:50 | 30:70 | 10:90 |
| 5/15 | 1/14 | 1/14 | 0/14 | 0/15 |
| (33%) | (7%) | (7%) | (0%) | (0%) |

n is the number of strains demonstrating bactericidal activity (defined as MBC:MIC ratio of ≤4); N is number of strains tested (strains with a starting MIC at the limit of detection could not be evaluated for bactericidal activity as an absolute fold-difference between MBC and MIC could not be determined)

CBA1 also demonstrated potent activity against M. genitalium, with similar MICs observed when tested at pristinamycin IA:flopristin ratios of 70:30 or 30:70 with both ratios providing MICs in the range of 0.015-0.12 μg/mL. Additionally, for both ratios tested, MIC values of CBA1 were 4- to 16-fold more potent than those observed with the benchmark comparator pristinamycin IA-pristinamycin IIA, which had MIC values ranging from 0.12-0.5 μg/mL, demonstrating superior potency of the CBA1 combination. When only the streptogramin A component (flopristin) of CBA1 was present, i.e., CBA1 at a ratio of 0:100, activity was also observed with an MIC range of 0.015-0.5 μg/mL against M. genitalium. Reduced activity was seen when only the streptogramin B component (pristinamycin IA) of CBA1 was present, i.e., CBA1 at a ratio of 100:0, which had an MIC range of 2-16 μg/mL.

### Example 3

### In Vitro Potency and Spectrum of Activity Compared to Known Streptogramin Combinations

The broader spectrum of antibacterial activity of CBA1 was evaluated against isolates of various gram-positive, gram-negative and atypical species and compared to known streptogramin combinations pristinamycin IA-pristinamycin IIA, quinupristin-dalfopristin, and/or linopristin-flopristin with all test articles at a streptogramin B:streptogramin A ratio of 30:70. The following species were evaluated: S. pneumoniae (30 isolates including 9 resistant to macrolides, 8 resistant to clindamycin, and 5 resistant to tetracyclines), Enterococcus faecium (30 isolates all resistant to vancomycin), Streptococcus pyogenes (30 isolates including 3 resistant to macrolides and 3 resistant to tetracyclines), Streptococcus agalactiae (30 isolates including 18 resistant to macrolides, 10 resistant to clindamycin, and 23 resistant to tetracyclines), S. aureus (96 clinical isolates collected from medical centers in the United States and Europe during 2019-2021 including 48 isolates resistant to methicillin and 12 resistant to clindamycin), N. gonorrhoeae (20 isolate panel of multi-drug resistant and extensively-drug resistant strains predominantly from the WHO and CDC Antimicrobial Resistance Isolate Bank collections), Mycoplasma genitalium (4 ATCC strains and 6 clinical isolates collected during 1980-2020 from the United Kingdom, Denmark, or the United States, including 3 with elevated MICs for azithromycin and moxifloxacin), Gardnerella vaginalis (30 clinical isolates collected during 1992-2020 from the United States and Italy), Bacillus anthracis (genetically diverse panel of 30 strains), Francisella tularensis (genetically diverse panel of 30 strains), Burkholderia mallei (panel of 10 strains), Burkholderia pseudomallei (panel of 10 strains), and Yersinia pestis (panel of 10 strains).

MICs were determined by broth microdilution as described in Example 2 following CLSI guideline documents M07 and M100 for S. aureus, Streptococcus spp., E. faecium, and N. gonorrhoeae, following CLSI methods for infrequently isolated or fastidious bacteria for B. anthracis, F. tularensis, Burkholderia mallei, Burkholderia pseudomallei, and Yersinia pestis (CLSI “Methods for Antimicrobial Dilution and Disk Susceptibility Testing of Infrequently Isolated or Fastidious Bacteria. 3^{rd} ed.” CLSI guideline 45. CLSI, 950 West Valley Road, Suite 2500, Wayne, Pennsylvania 19087 USA, 2016), and following CLSI guidelines for mycoplasmas (CLSI “Methods for antimicrobial susceptibility testing of human mycoplasmas” Approved Guideline M43-A. CLSI, 950 West Valley Road, Suite 2500, Wayne, Pennsylvania 19087 USA, 2011) for M. genitalium. For G. vaginalis, MICs were determined by agar dilution as recommended by CLSI for susceptibility testing of anaerobic bacteria (CLSI “Methods for Antimicrobial Susceptibility Testing of Anaerobic Bacteria, Approved Standard—Eleventh Edition” CLSI document M11, 11th Ed. CLSI, 950 West Valley Road, Suite 2500, Wayne, Pennsylvania 19087 USA, 2018). Supplemented Brucella agar plates containing 5% (v/v) laked sheep blood were utilized for susceptibility testing and plates were incubated in a Bactron 600 anaerobe chamber (Sheldon Manufacturing, Cornelius, Oregon) containing an atmosphere of 5% carbon dioxide, 5% hydrogen, and 90% nitrogen. MIC endpoints were determined at 72 hours for all G. vaginalis isolates with the MIC defined as the minimum concentration at which a marked reduction occurs in the appearance of growth on the test plate as compared to that of growth on the anaerobic control plate. For all species, CBA1, pristinamycin IA-pristinamycin IIA, quinupristin-dalfopristin, or linopristin-flopristin were all tested at a streptogramin B:streptogramin A ratio of 30:70.

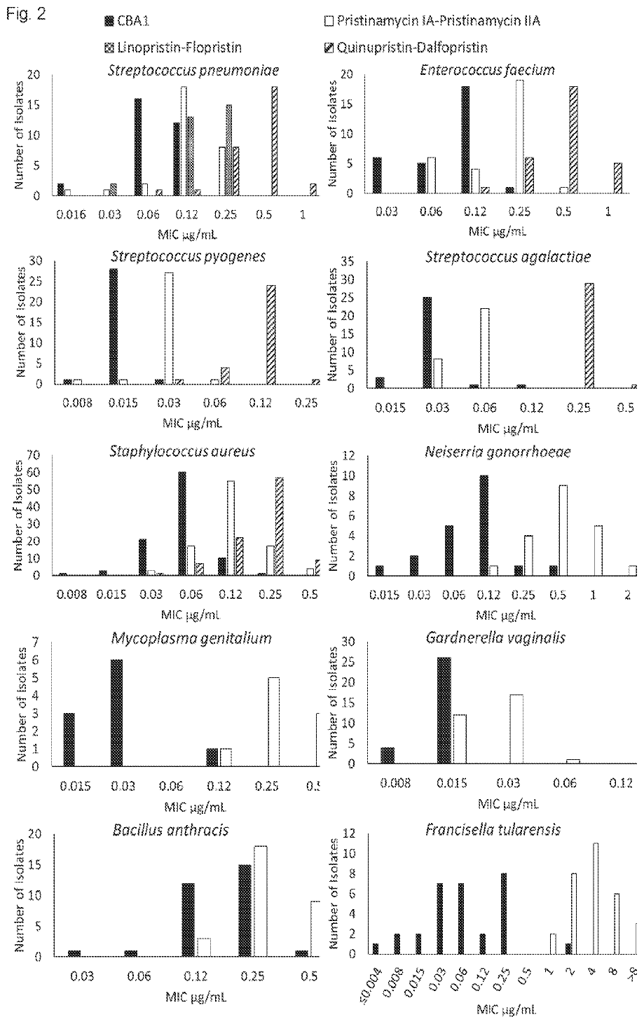

The MIC distributions for each species are presented as histograms in FIG. 2. The potency of the novel combination CBA1 over other known combinations is demonstrated in all species tested. Against each species, the MIC distribution for CBA1 was better than comparator streptogramin combinations pristinamycin IA-pristinamycin IIA, quinupristin-dalfopristin, or linopristin-flopristin with lower MICs obtained against almost all isolates evaluated as indicated by a shift in the MIC distribution to the left of the histograms. The MIC₉₀ values for CBA1 against S. pneumoniae, E. faecium, S. pyogenes, S. agalactiae, S. aureus, N. gonorrhoeae, M. genitalium, G. vaginalis, B. anthracis, and F. tularensis were 0.12, 0.12, 0.015, 0.03, 0.12, 0.012, 0.03, 0.015, 0.25, and 0.25 μg/mL, respectively (Table 15). When MIC₉₀ values are compared, CBA1 is more potent than the two known streptogramin combinations approved for human use, i.e., 2- to 32-fold more potent than pristinamycin IA-pristinamycin IIA and 2- to 8-fold more potent than quinupristin-dalfopristin across all species tested (Table 15).

CBA1 also demonstrated activity against B. mallei, B. pseudomallei, and Y. pestis, with an MIC range of 1-4 μg/mL, 4->8 μg/mL, and 1-4 μg/mL, respectively. For each species, the activity of CBA1 was 2- to 8-fold better than pristinamycin IA:pristinamycin IIA, which had MIC ranges of 4->8 μg/mL, all >8 μg/mL, and all 8 μg/mL, respectively.

TABLE 15
MIC₉₀ values against Various Bacterial Pathogens for CBA1 in Comparison to Those for
Pristinamycin IA-Pristinamycin IIA and Quinupristin-Dalfopristin

| | | | Pristinamycin IA- | | Quinupristin- | |
|---|---|---|---|---|---|---|
| | | | Pristinamycin IIA | | Dalfopristin | |
| | | | | Fold | | Fold |
| | | CBA1 | | increase | | increase |
| | | MIC₉₀ | MIC₉₀ | in Potency | MIC₉₀ | in Potency |
| Species | N | (μg/mL) | (μg/mL) | of CBA1 | (μg/mL) | of CBA1 |
| Streptococcus pneumoniae | 30 | 0.12 | 0.25 | 2 | 0.5 | 4 |
| Enterococcus faecium | 30 | 0.12 | 0.25 | 2 | 1 | 8 |
| Streptococcus pyogenes | 30 | 0.015 | 0.03 | 2 | 0.12 | 8 |
| Streptococcus agalactiae | 30 | 0.03 | 0.06 | 2 | 0.25 | 8 |
| Staphylococcus aureus | 96 | 0.12 | 0.25 | 2 | 0.25 | 2 |
| Neisseria gonorrhoeae | 20 | 0.12 | 1 | 8 | ND | ND |
| Mycoplasma genitalium | 10 | 0.03 | 0.5 | 16 | ND | ND |
| Gardnerella vaginalis | 30 | 0.015 | 0.03 | 2 | ND | ND |
| Bacillus anthracis | 30 | 0.25 | 0.5 | 2 | ND | ND |
| Francisella tularensis | 30 | 0.25 | 8 | 32 | ND | ND |

### Example 4

### In Vitro Time-Kill Kinetics

An in vitro time-kill assay allows a quantitative assessment of the bactericidal activity of an antibiotic at various timepoints following exposure to the antibiotic. The time-to-kill kinetics of the biological activity of CBA1 was evaluated against multi-drug resistant N. gonorrhoeae strains NCTC 13480 and NCTC 13820. Both of these strains are resistant to penicillin, tetracycline, and ciprofloxacin. NCTC 13480 has an elevated MIC for the only remaining standard of care antibiotic for the treatment of gonorrhoeae, meeting the CDC's Gonococcal Isolate Surveillance Project (GISP) “alert value” criteria. NCTC 13820 has high level resistance to both ceftriaxone and cefixime.

Baseline MIC values were measured in triplicate using the broth macrodilution (tube) methodology according to CLSI M07 guidelines (CLSI “Methods for Dilution Antimicrobial Susceptibility Tests for Bacteria That Grow Aerobically; Approved Standard—11th Edition” CLSI standard M07. CLSI, 950 West Valley Road, Suite 2500, Wayne, Pennsylvania 19087 USA, 2018). Fastidious broth was used as the testing media with a 4 mL total volume in each tube. Isolates were incubated for 24 hours at 35° C. in a 5% CO₂ incubator for all compounds. MIC values corresponded to the lowest concentration tube with a lack of turbidity upon visual examination.

Time-kill bactericidal activity assays were conducted according to methods described by CLSI (CLSI “Methods for determining bactericidal activity of antimicrobial agents; approved guideline. Document M26-A” CLSI, 950 West Valley Road, Suite 2500, Wayne, Pennsylvania 19087 USA, 1999). The strains were initially grown for 2 hours in in Fastidious Broth. Cultures were then diluted into tubes containing antimicrobial agents at 2×, 4×, and 8× the concentration of the modal baseline MIC in 4 mL of fastidious broth. Each testing event also included a growth control tube with no antimicrobial compound for each strain.

A sample of each tube was removed at 0, 2, 4, 8, and 24 hours of growth at 35° C. after the addition of the compound. Each sample was serial diluted in saline and plated on GC agar with 1% Gonococcus-Haemophilus influenzae (GCHI) enrichment for CFU measurements.

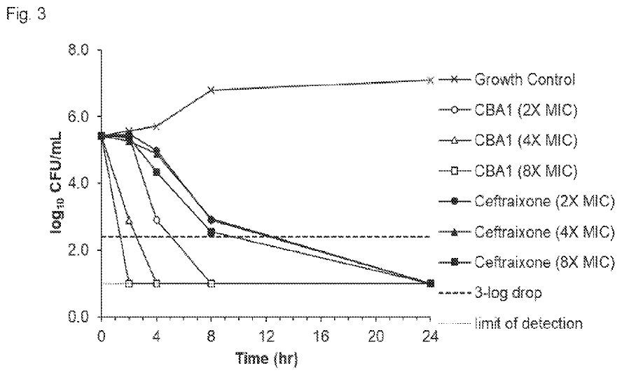

Results for NCTC 13480 are shown in FIG. 3. CBA1 resulted in bactericidal activity (i.e., ≥3-log₁₀ CFU/mL) within 2, 4, and 8 hours of exposure at 8×, 4×, and 2× the MIC, respectively. In contrast, the standard of care antibiotic for gonorrhea, ceftriaxone, did not achieve bactericidal activity until 24 hours of exposure, even at concentrations up to 8× the MIC. Similar results were observed when CBA1 was evaluated against N. gonorrhoeae strain NCTC 13820 with bactericidal activity occurring within 2 hours at 8× and 4× the CBA1 MIC and within 4 hours at 2× the CBA1 MIC. These results demonstrate that the novel streptogramin combination CBA1 possesses the desirable property of rapid bactericidal activity.

### Example 5

### In Vivo Efficacy

Animal infection models hold a high value for proof of concept of in vivo efficacy in antibiotic development. Unlike most other therapeutic areas, in the case of antibiotics, the drug target is the bacteria, not the host. The murine neutropenic thigh infection model is one of the most commonly used models to evaluate in vivo antibacterial efficacy. The primary endpoint in this model is the reduction in bacterial CFU at 24 hours after initiation of antibiotic therapy; and this endpoint in mice has been shown to correlate with clinical outcomes in human subjects.

The in vivo efficacy of CBA1 administered via intravenous or oral routes against S. aureus was evaluated in a murine neutropenic thigh infection model using MRSA strain BAA-1717. For the intravenous study, female CD-1 mice from Charles River Laboratories were rendered neutropenic via two doses of cyclophosphamide on days −4 and −1 with 150 mg/kg and 100 mg/kg delivered via intraperitoneal injection, respectively. On study Day 0, mice were infected with methicillin-resistant S. aureus strain ATCC BAA-1717 by injection of 100 μL of the prepared bacterial inoculum (1.9×10⁵ CFU/mouse thigh) into the right thigh muscle. Groups of 4 mice were administered either vehicle (5% DMSO, 25% PEG-400, 30% propylene glycol and 40% saline) or test agents twice via tail vein injection at 2 and 14 hours post-infection with the dose of agents administered based on body weight at 5 mL/kg. Test agents included CBA1 at 15 mg/kg for each dose (a 33:67 weight ratio of streptogramin B:streptogramin A), quinupristin-dalfopristin at 15 mg/kg for each dose (at a 30:70 ratio of streptogramin B:streptogramin A), and positive control vancomycin at 50 mg/kg for each dose. The MICs of CBA1, quinupristin-dalfopristin, and vancomycin against the BAA-1717 strain were 0.06, 0.12 and 1 μg/mL, respectively.

Vehicle infection control groups were euthanized at 2 and 26 hours post-infection and test agent groups were euthanized at 26 hours post-infection. The right thigh from each animal was aseptically explanted, weighed, and homogenized to a uniform consistency. Homogenized samples were serially diluted and plated on bacterial growth media for CFU determination. CFUs were enumerated after overnight incubation at 37° C. The CFUs were adjusted for dilution and tissue weight for each thigh with averages and standard deviation calculated for each group.

The same study was performed to evaluate efficacy of test agents administered by oral dosing in neutropenic mice infected with S. aureus strain BAA-1717. In this study, groups of 4 mice were administered either vehicle (0.5% methylcelluose (4000 CP), 0.2% Tween 80) or test agents twice at 2 and 14 hours post-infection via oral gavage with dose volume based on body weight at 10 mL/kg. Test agents included CBA1 at 75 mg/kg for each dose or pristinamycin IA-pristinamycin at 150 mg/kg for each dose, both at a 30:70 ratio of streptogramin B:streptogramin A, and the positive control linezolid was dosed at 100 mg/kg for each dose. The MICs of CBA1, pristinamycin IA-pristinamycin IIA, and linezolid against the BAA1717 strain were 0.06, 0.12 and 2 μg/mL, respectively. At 2 hours or 26 hours post-infection, thighs were processed and CFUs were determined as described above.

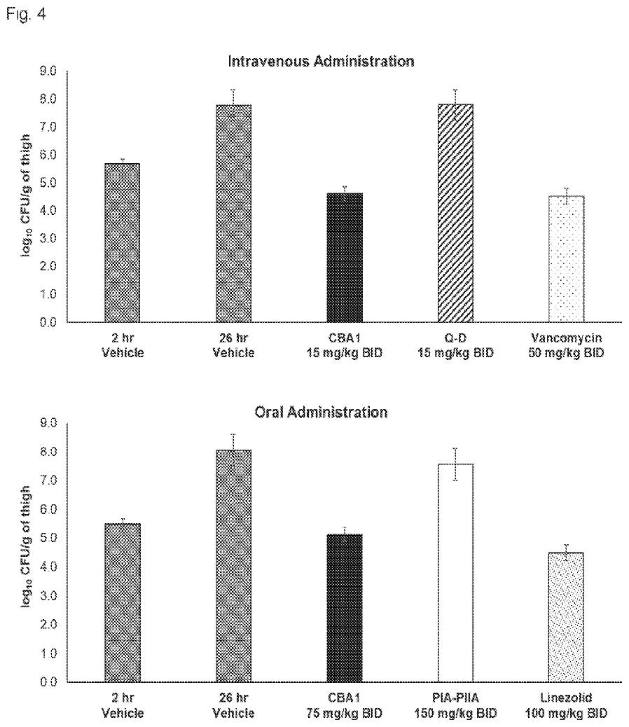

Following intravenous administration of 15 mg/kg twice (at 2 and 14 hours post-infection), CBA1 demonstrated superior efficacy compared to quinupristin-dalfopristin administered with the same dose and regimen and was comparable to standard of care vancomycin administered intravenously at a 3.3-fold higher dose (50 mg/kg twice). Results are shown in FIG. 4 (top) as the average log₁₀ CFU/g of thigh tissue±standard deviation. At 26 hours post-infection, CBA1 reduced the bacterial load by 3.17 log₁₀ CFU compared to vehicle control whereas quinupristin-dalfopristin failed to have an impact on bacterial load (+0.01 log₁₀ CFU compared to vehicle) when administered at the same dose level as CBA1.

Following oral administration of 75 mg/kg twice (at 2 and 14 hours post-infection), CBA1 demonstrated superior efficacy compared to pristinamycin IA-pristinamycin IIA administered orally at a 2-fold higher dose (150 mg/kg twice) and comparable to standard of care linezolid administered orally at a 1.3-fold higher dose (100 mg/kg twice). Results are shown in FIG. 4 (bottom) as the average log₁₀ CFU/g of thigh tissue±standard deviation. At 26 hours post-infection, CBA1 reduced the bacterial load by 2.92 CFU/mL log₁₀ CFU compared to vehicle control whereas pristinamycin IA-pristinamycin IIA only reduced bacterial load by 0.47 log₁₀ CFU even though it was administered at twice as high as the dose of CBA1.

Collectively these in vivo data demonstrate the significantly improved in vivo efficacy of the novel, unique streptogramin combination CBA1 (pristinamycin IA+flopristin) over the known streptogramin antibiotics approved for human use. The enhanced in vivo efficacy of CBA1 over existing streptogramin combinations are supported by its superior antibacterial potency and desirable antibacterial profiles, including its bactericidal activity, as evidenced from the vast amount of in vitro data presented herein.

### Other Embodiments

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Unless otherwise indicated, all numbers expressing a characteristic, item, quantity, parameter, property, term, and so forth used in the present specification and claims are to be understood as being modified in all instances by the term “about.” Unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only and is not intended to limit the scope of the present invention, which is defined solely by the claims. Accordingly, the present invention is not limited to that precisely as shown and described.

1.-32. (canceled)

33. A composition comprising pristinamycin IA (PIA) or a pharmaceutically acceptable salt thereof; and flopristin or a pharmaceutically acceptable salt thereof.

34. The composition of claim 33 wherein the PIA:flopristin ratio is in the range from about 1:99 to about 99:1 by weight.

35. A kit or a packaged article, comprising a first composition comprising pristinamycin IA or a pharmaceutically acceptable salt thereof; and a second composition comprising flopristin or a pharmaceutically acceptable salt thereof; and instructions for administering the first composition and the second composition in a method for treating an infection caused at least in part by a bacterium in a subject.

36. The kit or packaged article of claim 35 wherein the instructions include administering to the subject the first composition and the second composition simultaneously, administering the first composition before the second composition is administered, or administering the first composition after the second composition is administered.

37. A method for treating or preventing a bacterial infection in a subject, the method comprising administering a therapeutically effective amount of the composition of claim 33, or providing a kit or packaged article comprising a therapeutically effective amount of pristinamycin IA or a pharmaceutically acceptable salt thereof; and a therapeutically effective amount of flopristin or a pharmaceutically acceptable salt thereof to the subject.

38. The method of claim 37 wherein the infection is a sexually transmitted infection or a genitourinary tract infection.

39. The method of claim 38 wherein the infection is caused at least in part by one or more strains of Neisseria gonorrhoeae, Chlamydia trachomatis, Mycoplasma genitalium, Mycoplasma hominis, Ureaplasma urealyticum, Ureaplasma parvum, or Gardnerella vaginalis, or a combination thereof.

40. The method of claim 37 wherein the infection is a respiratory infection.

41. The method of claim 40 wherein the respiratory infection is a community acquired pneumonia, healthcare associated pneumonia, chronic bronchitis, sinusitis, acute maxillary sinusitis, acute exacerbations of chronic bronchitis, pharyngitis, chronic obstructive pulmonary disease, or non-tuberculosis mycobacteria infection.

42. The method of claim 40 wherein the respiratory infection is caused at least in part by one or more strains of Streptococcus pneumoniae, Streptococcus pyogenes, Staphylococcus aureus, Haemophilus influenzae, Haemophilus parainfluenzae, Moraxella catarrhalis, Legionella spp., Chlamydia pneumoniae, Mycoplasma pneumoniae, Bordetella pertussis, Neisseria gonorrhoeae, Corynebacterium diphtheriae, Coxiella burnettii, Peptostreptococcus spp., Fusobacterium spp., Bacteroides spp., Prevotella spp., Nocardia spp., or Mycobacterium spp., or a combination thereof.

43. The method of claim 40 wherein the subject has cystic fibrosis.

44. The method of claim 37 wherein the infection is an infection of the skin, soft tissues, bones, or joints, or a combination thereof.

45. The method of claim 44 wherein the infection is caused at least in part by one or more strains of Staphylococcus aureus, Staphylococcus haemolyticus, Staphylococcus lugdunensis, Streptococcus agalactiae, viridans group streptococci, Streptococcus pyogenes, Salmonella spp., Shigella spp., Campylobacter spp., Enterococcus faecalis, Enterococcus faecium, Corynebacterium spp., Propionibacterium acnes, Kingella kingae, Chlamydia trachomatis or Neisseria gonorrhoeae, or a combination thereof.

46. The method of claim 37 wherein the subject has endocarditis caused at least in part by or associated with the bacterial infection.

47. The method of claim 46 wherein the infection is caused at least in part by one or more strains of Staphylococcus spp., Streptococcus spp., Enterococcus spp., Chlamydia spp., Haemophilus spp., Kingella spp., Legionella spp., Corynebacterium spp., Listeria monocytogenes, or Neisseria gonorrhoeae, or a combination thereof.

48. The method of claim 37 wherein the subject has bacteremia or sepsis caused at least in part by or associated with the bacterial infection.

49. The method of claim 37 wherein the subject has anthrax, tularemia, plague, glanders or meliodosis caused at least in part by or associated with the bacterial infection.

50. The method of claim 49 wherein the infection is caused at least in part by one or more strains of Bacillus anthracis, Francisella tularensis, Yersinia pestis, Burkholderia mallei, or Burkholderia pseudomallei, or a combination thereof.

51. The method of claim 37 wherein the subject is an animal or a human.

52. A composition comprising quinupristin or a pharmaceutically acceptable salt thereof; and flopristin or a pharmaceutically acceptable salt thereof.
